(19) United States
(12) Patent Application Publication
(10) Pub. No.: US 2004/0058357 A1
(43) Pub. Date: Mar. 25, 2004

(54) System and method for assaying drugs

(76) Inventors: Hermona Soreg, Jerusalem (IL); Eran Meshorer, Jerusalem (IL); Ella Sklan, Rehovot (IL); Shai Shoham, Jerusalem (IL)

Correspondence Address: Cooper & Dunham, 1185 Avenue of the Americas, New York, NY, 10036, US

(21) Appl. No.: 10/432,131

(22) PCT Filed: Nov. 14, 2001
(86) PCT No.: PCT/IL01/01051

(51) Int. Cl. C12Q 1/68

(52) U.S. Cl. 435/6

(57) ABSTRACT

The invention relates to a method and system for evaluating an effect on the nervous system of a test drug by comparing the effect of such drug on AChE catalytic activity or isoform variance in the brain of a test animal following challenge by an AChE blocker (e.g. DFP) or a blocker of AChE and muscarinic receptors M1 and M2 (e.g. pyridostigmine) and comparing this effect with that of a known agent, preferably a non-selective muscarinic receptor blocker (e.g. scopolamine) or a specific M1 receptor blocker (e.g. pirenzepine). Also provided is a method of screening for a candidate drug that is a modulator of the expression of any one of AChE variants and isoforms by determining the effect of such drug on the translocation of an AChE isoform within a neuron. Further provided is a method of screening for a candidate drug aimed at affecting central nervous system properties which is a modulator of the interaction between AChE-R/RACK1/PKC.

### FIELD OF THE INVENTION

The invention relates to the field of drug screening and evaluation, particularly the evaluation of the effect of drugs on the central nervous system.

### BACKGROUND OF THE INVENTION

Physiological stress induces rapid and robust signaling processes in mammalian brain neurons, mainly through the cholinergic system. The molecular pathway(s) which enable the adjustment to stressful stimuli are not yet fully understood. Drugs that interfere with the cholinergic system are increasingly used for treatment of stress-related conditions as well as neurodegenerative diseases, but the effects of such treatment on cholinergic functions have not been thoroughly analyzed at the molecular level. The pre-clinical testing of neuroactive drugs is imperative for a complete assessment of the outcome of their clinical use in patients with compromised cholinergic neurotransmission. The present invention provides a platform for pre-clinical testing of such drugs.

Acetylcholine-mediated neurotransmission is fundamental for nervous system function. Acetylcholinesterase (AChE), a type B carboxylesterase, hydrolyses and inactivates acetylcholine (ACh), thereby regulating the concentration of the transmitter at the synapse. Termination of activation is normally dependent on dissociation of ACh from the receptor (AChR) and its subsequent diffusion and hydrolysis, except in diseases where ACh levels are limiting or under AChE inhibition, conditions that increase the duration of receptor activation [Soreq, H. and S. Seidman. (2001) Nat Rev Neurosci 2:294-302].

AChE has three splice variants (FIG. 1). The “synaptic” form (AChE-S) is the principal AChE mRNA transcript in brain and muscle tissues, and is formed by joining exon 4 to exon 6, which results in a protein with an amphipathic C-terminus. The second most common transcript, the AChE “erythrocytic” isoform (AChE-E), links exons 4 and 5 which encode a 43 amino acid C-terminal peptide. The rare “readthrough” transcript (AChE-R), is expressed in embryonic and tumor cells [Karpel, R. et al. (1994) Exp Cell Res 210:268-77], and is produced by uninterrupted transcription through intron 4, resulting in a peptide containing a hydrophilic C-terminal extension of 26 amino acids [Li, Y. et al. (1991) J Biol Chem 266:23083-90]. AChE-R is overproduced in response to psychological stress, AChE inhibition or following head trauma [Cohen, O. et al. (2001) Mol Psych; Kaufer, D. et al. (1998) Nature 393:373-7; Shohami, E. et al. (2000) J. Mol. Med. 78:228-236].

Stimulus-induced changes in alternative splicing have recently emerged as a major mechanism of neuronal adaptation to stress, contributing to the versatility and complexity of the expression patterns of at least 40% of the genes in the human genome [Modrek, B. et al. (2001) Nucleic Acids Res 29, 2850-9]. Examples of stress-induced changes in alternative splicing include the glucocorticoid receptor McCormick, J. A et al. (2000) Mol Endocrinol 14, 506-17), amongst others. Another stimulus-induced post-transcriptional process is dendritic mRNA translocation [Davis, L. et al. (1987) Nature 330, 477-9], that has been observed in numerous transcripts [Steward, O. and E. M. Schuman (2001) Annu Rev Neurosci 24, 299-325], and whose consequences may impair synaptic plasticity [H. Kang and E. M. Schuman (1996) Science 273, 1402-6] and long-term memory [Bailey, C. H. et al. (1996) Proc Natl Acad Sci USA 93, 13445-52].

Changes in the level and mode of AChE gene expression are revealing indicators of alteration in cholinergic neurotransmission. For example, both acute psychological stress and exposure to organophosphate and carbamate AChE inhibitors (anti-AChEs) were found to induce rapid, yet long-lasting transcriptional AChE activation that was accompanied by a splicing shift, from the major ACHE-S variant to the rare AChE-R mRNA and protein [Kaufer (1998) id ibid.; Soreq & Seidman (2001) id ibid.]. Subsequent studies have indicated that these processes are at least in part induced by glucocorticoid hormones [Shapira, M. et al. (2000) Hum Mol Genet 9:1273-1281] and involve muscarinic receptor activation [Nitsch R M et al. (1998) J Physiol Paris 92(3-4):257-64; Anguelova, E. et al. (2000) J Neurosci Res 59(2):209-17].

Muscarinic acetylcholine receptors (mAChRs) belong to the class of receptors coupled to guanidine-dependent proteins (G-proteins), that intracellularly trigger a signal transduction cascade through the activation of phospholipase C (PLC). There are at least five isoforms of mAChRs (M1-M5), which are discernible pharmacologically by their interaction with specific antagonists. All mAChR subtypes are present in the cortex and hippocampus, whereas M2 is the major form in the medulla and heart [Soreq, H. and Zakut, H. Human cholinesterases and anticholinesterases. 1 ed. Academic Press, San Diego, 314 pp. (1993)]. Selective blockade of the M2 receptor was shown to facilitate ACh release while improving cognition [Lachowicz, J. et al (2001) Life Sci 68(22-23):2585-92], suggesting that this presynaptic inhibitory receptor blocks the feedback response by limiting ACh outflow. Moreover, exposure to pyridostigmine, another anticholinesterase drug, was shown to induce neuronal apoptosis mediated by muscarinic receptors [Gunasekar, P. et al. (2000) J Neurosci Res 61(5):570-5]. It is therefore of special interest to focus on muscarinic receptors and their involvement with stress and/or anti-AChE responses in the context of changes in ACHE gene expression

Ample information suggests the involvement of specific protein kinases in at least some of these stress-induced processes. The enzymatic activity of certain subtypes of protein kinase C (PKC) [Coussens, L. et al. (1986) Science, 233, 859-66] was shown to be subject to changes (i.e. biochemical activation, membrane translocation) under physiological [Hu, G. Y. et al. (1987) Nature, 328, 426-9], biochemical [Macek, T. A. et al. (1998) J Neurosci, 18, 6138-46] and cytoarchitectural [Tint, I. S. et al. (1992) Proc Natl Acad Sci USA, 89, 8160-4] responses at the cellular and organismal levels. Of the many subtypes of PKC, PKCβII appears more directly relevant than others to physiological stress responses. PKCβII undergoes specific activation and membrane translocation under ischemia [Cardell, M. and Wieloch, T. (1993) J Neurochem, 61, 1308-14]. Intracellularly, PKCβII molecules are translocated to those neuronal perikaryon sites where they function with the aid of RACK1, the intracellular receptor for activated protein kinase C [Ron, D. et al. (1999) J Biol Chem, 274, 27039-46]. However, RACK1, like PKCβII, is ubiquitously present in numerous cell types, which makes it unlikely to be the primary inducer of PKCβII-mediated stress related cascades. A relevant putative mediator of the stress-related changes in PKC activities should be intracellular in its location and capable of activating or translocating active PKC within neuronal perikarya. The “readthrough” acetylcholinesterase variant AChE-R is a promising candidate for this role [Soreq, H. and Seidman, S. (2001) Nat Rev Neurosci, 2, 294-302].

Using a yeast two-hybrid screen, the inventors have previously discovered that the C-terminal domain of ACHE-R forms a tight complex with RACK1 [PCT/IL00/00311]. Therefore, the potential involvement of AChE-R in PKCβII stress induced signaling pathways have been further investigated. The present invention demonstrates that ACHE-R, RACK1 and PKCβII, can be co-immunoprecipitated. Moreover, AChE-R interaction activates PKCβII and facilitates its translocation into densely packed neuronal clusters, which may be causally involved with the stress-protection capacity of overexpressed AChE-R.

High AChE levels were reported in astrocytomas and the more aggressive glioblastoma tumors, which usually develop in pre-existing astrocytoma [Barbosa, M. et al. (2001) Surg. Neurol. 55:106-12; Razon, N. et al. (1984) Exp. Neurol. 84:681-695]. Astrocytomas are graded into several subclasses of increasing invasiveness associated with distinct morphologies, which may assist in searches for tumor-affecting genes [Daumas-Duport, C. et al. (1988) Cancer 62:2152-65]. AChE is a particularly attractive candidate for such a role, since AChE levels are very low in all types of normal glia [Karpel (1996) id ibid.; Soreq, H. et al. (1984) EMBO J. 3:1371-5], but increase in tumors. This further suggests the involvement of tumor-specific transcription factors regulating AChE gene expression. While this is compatible with the recently established morphogenetic activities of ACHE [Soreq, H. and S. Seidman (2001), id ibid.], the molecular origin(s) of AChE possible tumorigenic involvement is still unknown. The inventors have found that expression of the transcriptional activator Runx1/AML, well known for its role in leukemia, is increased in initial stages of astrocytoma, and correlates with the subsequent induction of AChE variants (see Example 4 of the present application).

The rapidly increasing use of anti-cholinesterases (anti-ChEs) for treatment of Alzheimer's disease (AD) is currently limited to patients with moderate to severe symptoms. AD patients treated with anti-AChEs display elevated AChE activities in their cerebrospinal fluids, suggesting that AChE overexpression is a normal human response to such inhibitors [Nordberg, A. et al. (1999) Alzheimer's Reports 2(6), 347-352]. However, it is not yet known which AChE variant accumulates in these patients' brains and whether their impaired cholinergic neurotransmission alters the feedback response to anti-AChE exposure. Understanding the pathways leading to AChE over-expression and shifted alternative splicing is, further, important prophylactic [Friedman, A. et al. (1996) Nat Med 2:1382-1385] treatment with an anti-AChE, as well as in cases of occupational exposure, e.g. to agricultural insecticides [Mackey C (1982) Heart Lung 11(5):479-84] or under terror attacks or warfare conditions, e.g. for Gulf Wax Veterans [Sapolsky R M.(1998) Nature Med 393:308-309]. On another level, recent studies with AChE overexpressing transgenic mice suggested that the initial feedback response of AChE overproduction might lead to a secondary compensation of increased ACh release [Erb C. et al. (2001) J Neurochem 77(2):638-46], emphasizing the need for careful follow-up of the kinetics of these responses. Also, transgenic AChE-S lead to progressive deterioration of learning and memory [Beeri R. et al. (1995) Curr Biol 5(9):1063-71], neuromuscular malfunction [Andres, C. et al. (1997) Proc Natl Acad Sci USA 94(15):8173-8], cumulative neuronal stress markers [Sterfeld, M. et al. (2000) Proc. Natl. Acad. Sci. USA 97(15):8647-8652] with excessive high affinity choline transport [Erb et al. (2001) id ibid.]. In contrast, transgenic human AChE-R protected the mouse brain from accumulating aging associated neuropathology hallmarks of stress [Sternfeld et al. (2000) id ibid.], yet caused impaired spermatogenesis and reduced fertility Nor, I. et al. (2001) FASEB J.

AD is notably associated with progressive loss of cholinergic neurons [Coyle, (1983)], and several reports indicate effects of these drugs in patients at the more advanced phases of neurodegeneration. However, the effects of cholinergic malfunction on organismal responses to inhibitors of acetylcholinesterase (ACHE) have not been systematically dissected. This is of special importance because various external stimuli, including, but not limited to, exposure to AChE inhibitors, induce dynamic changes in the level and composition of AChE isoforms, but it remained unclear whether and to what extent these changes depend on the integrity of cholinergic neurotransmission in the brain of the treated organism.

Therefore, in search for a system that would better reflect the full extent of the effects of drugs on the integrity of cholinergic neurotransmission in the brain of the treated organism, particularly those upstream and downstream of AChE activity, the inventors have developed a multi-parametric method for evaluating the effects of manipulation of muscarinic neurotransmission on anti-ChE responses in the rat brain.

It is therefore an object of this invention to provide a method for evaluating an effect of a test drug on the nervous system, particularly on the cholinergic system. It is another object of this invention to provide a screening method for a candidate drug that is a modulator of the expression of AChE. It is yet a further object of this invention a screening method for a candidate drug which is a modulator of the interaction between AChE/RACK1/PKC.

These and other objects of the invention will be elaborated on as the description proceeds.

### SUMMARY OF THE INVENTION

The invention relates to a method and system for evaluating an effect on the nervous system of a test drug by comparing the effect of such drug on AChE catalytic activity or isoform variance in the brain of a test animal following challenge by an AChE blocker (e.g. DFP) or a blocker of AChE and muscarinic receptors M1 and M2 (e.g. pyridostigmine) and comparing this effect with that of a known agent, preferably a non-selective muscarinic receptor blocker (e.g. scopolamine) or a specific muscarinic 1 receptor blocker (e.g. pirenzepine).

More particularly, the present invention relates to a method for evaluating an effect on the nervous system of a test drug, wherein said method comprises the steps of:

(a) providing the following groups of animals: (i) at least one control group; and (ii) a identical number of test groups;

(b) at time zero, injecting each of the animals of the control group with a carrier and each of the animals of the test group with said test drug contained within said carrier;

(c) after a predetermined period of time from time zero, injecting the animals of the control group with an agent selected from:

said carrier, an irreversible acetylcholinesterase (AChE) blocker or an AChE, M1 and M2 blocker; and

injecting the animals of the test group with the same agent used for the control group;

(d) after a first predetermined period of time from step (c), sacrificing a number of animals from each of said control and test groups and dissecting their brains;

(e) after a second predetermined period of time from step (c), sacrificing the remaining of animals of each group and dissecting their brains;

(f) subjecting the dissected brains to at least one analytical procedure for assessing AChE catalytic activity or isoform variance, or any combination thereof;

(g) generating a drug profile from the results of the analytical procedures performed in (f) for each of the control and test groups; and

(h) comparing the profile of the test group with the profile displayed by the control groups;

whereby

increased expression in the brain of AChE mRNA transcripts, with or without a shift in the alternative splicing of said transcripts from the primary S to the normally rare R variant indicates a distinct feedback response to said test drug;

an increase with time in the catalytic activity of brain AChE, with or without an increase in the globular GI AChE monomers to G4 tetramers reflects the outcome of such feedback at protein level; and

any diversion from the brain response of the control to said irreversible AChE blocker reflects an impairment in muscarinic neurotransmission resulting from the exposure to said test drug.

In a preferred embodiments, the method of the invention employs in step (b), three test groups and the following three corresponding control groups:

group (i-a), wherein at time zero each of the animals is injected with a carrier, providing baseline control;

group (i-b), wherein at time zero each of the animals is injected with a general non-selective muscarinic receptor blocker; and

group (i-c), wherein at time zero each of the animals is injected with a M1 blocker; and in step (c) injecting the rats of the control group with:

a carrier, in case a carrier or a general non-selective, muscarinic receptor blocker or M1 blocker was used in step (b);

an acetylcholinesterase (AChE) blocker, in case a carrier or a general muscarinic receptor blocker or M1 blocker was used in step (b); or

an AChE, M1 and M2 blocker, in case a carrier was used in step (b); and each test group is injected with the same agent injected to the corresponding control group.

In the method of the invention, said predetermined period of time from time zero is from 45 to 90, preferably 45 minutes, said first predetermined period of time from step (c) is from 2 to 4, preferably 3 hours, and said second predetermined period of time from step (c) is from 24 to 36, preferably 24 hours.

The test animals are preferably rats.

A preferred general non-selective muscarinic receptor blocker is scopolamine, a preferred selective M1 blocker is pirenzepine, a preferred AChE blocker is DFP, a preferred AChE, M1 and M2 blocker is octylpyridostigmine (OP) and a preferred carrier is propylene glycol/saline.

In the method of the invention, the analytical procedures are preferably selected from the group consisting of determining AChE activity, determining ACHE S and/or R isoforms ratio and determining AChE-S and/or -R mRNA variants tissue expression and distribution, by any suitable means, for example by means of a sucrose gradient.

The AChE isoform tissue expression may be determined by any one of in situ hybridization utilizing isoform specific probes, and immunohistochemistry utilizing isoform specific antibodies.

The drugs to be evaluated by the method of the invention can be any candidate or known drugs, e.g. drugs for the treatment of anxiety conditions, post-traumatic stress, Alzheimer's disease, muscle malfunctioning, neuroclegenerative disorders, damage resulting from exposure to xenobiotics, panic, neuromuscular disorders, Parkinson's disease, Huntington's chorea, muscle fatigue, multiple chemical sensitivity, autism, multiple sclerosis and Shorgren's disease.

The method of the invention further enables to evaluate the astrocyte oncogenetic potential of a test drug, preferably by the following steps:

(a) treating an animal with said test drug;

(b) dissecting the brain of said animal;

(c) contacting said dissected brain with detectable means for determining the expression of Runx1/AML;

(d) comparing the level of expression of RunxI/AML in said brain with the level of expression of the same gene in a brain tumor sample.

In a further aspect, the invention provides method of screening for a candidate drug that is a modulator of the expression of any one of AChE variants and isoforms, wherein said screening method comprises the steps of:

(a) contacting a cell, preferably a neuron, with a test drug under suitable conditions;

(b) determining the effect of said drug on an end-point indication, wherein said effect is the capacity of a drug to modulate the expression and distribution of at least one of the AChE isoforms.

In this screening method, the endpoint indication may be the translocation of an AChE isoform within the cell, particularly a neuron, more particularly translocation is from the neuron body towards its processes.

The said AChE isoform expression and distribution may be determined by in situ hybridization, whereby the detection of AChE expression and distribution is performed with ACHE mRNA variant specific probes.

Further, in this screening method the cells to be treated may be selected from the group consisting of PC12 cells and primary cerebellar neurons or any AChE-expressing primary neuron or established cell line.

In yet a further embodiment, the invention relates to another method of screening for a candidate drug aimed at affecting central nervous system properties which is a modulator of the interaction between AChE-R/RACK1I/PKC, said drug to be further evaluated by the method of the invention, which further screening method comprises the steps of:

a. providing a reaction mixture comprising the AChE-R variant of AChE or any functional fragment thereof, the cognate receptor for activated kinase C(RACK1) and the protein kinase C βII (PKCβII);

b. contacting said mixture with a test drug under suitable conditions for said interaction; and

c. determining the effect of the test drug on an end-point indication, wherein said effect is indicative of modulation of said interaction by the test drug.

In this further screening method, the said modulator inhibits or enhances the interaction between AChE-R/RACK1I/PKC.

The reaction mixture may be a cell mixture or a cell-free mixture, and may optionally further comprises solutions, buffers and compounds which provide suitable conditions for interaction between AChE-R/RACK1/PKC and the detection of an end-point indication for said interaction.

In this further screening method, the modification of said end-point indicates modulation of the interaction between AChE-R/RACK1/PKC by said test drug.

In one embodiment of this further screening method, the reaction mixture is a cell-free mixture.

In this embodiment, the further screening method comprises the steps of:

a. providing a cell free mixture comprising the AChE-R variant of ACHE or any functional fragment thereof, RACK1 and PKCβII;

b. contacting said mixture with a test drug under conditions suitable for an in vitro interaction; and

c. determining the effect of the test substance on co-precipitation of PKCβII and RACK1 with the ACHE-R or fragment thereof as an end-point indication, whereby the absence or increase of said co-precipitation indicates modulation of formation of a complex between AChE-R/RACK1/PKC by the test drug.

The cell-free mixture may comprise any one of AChE-R variant of AChE or any functional fragment thereof, RACK1 and PKCβII, which are provided as a purified recombinant protein or as a cell lysate of cell expressing said proteins.

The said ACHE-R variant of AChE, may be a fusion protein comprising AChE-R or functional fragment thereof and any one of GST (Glutathion-S-Transferase) and GFP (Green Fluorescent Protein).

In another embodiment of this further screening method, the said reaction mixture is a cell mixture, particularly a transfected cell culture, and more particularly transfected mammalian cell culture.

In this embodiment, the further screening method comprises the steps of:

a. providing transfected cell culture expressing the AChE-R variant of AChE or functional fragment thereof, the cognate receptor for activated kinase C (RACK1) and the PKCβII;

b. contacting said transfected cell culture with a test substance;

c. detecting the interaction between AChE-R/RACK1/PKC in the presence of the test substance/drug by searching for an end-point indication, whereby inhibition of said end-point indicates inhibition of complex formation between AChE-R/RACKl/PKC by said test drug.

The transfected cell to be used may be transfected by:

a. an expression vector comprising a nucleotide sequence coding for the AChE-R variant of AChE or a functional fragment thereof;

b. optionally, constructs comprising a nucleic acid sequence coding for any one of the cognate receptor for activated kinase C (RACK1) and the PKCβII.

The end-point indication may be the subcellular translocation of catalytically active PKCβII, which can be detected by a visually detectable signal.

Alternatively, the end-point indication may be co-precipitation of PKCβII and RACK1 with the AChE-R or functional fragment thereof leading to a detectable signal, whereby modification of said detectable signal in the presence of the test drug indicates modulation of the formation of a complex between AChE-R/RACK1/PKC by said test drug.

The evaluation and screening methods of the invention are suitable for assessing and screening for any test drug, e.g. test drugs selected from protein based, carbohydrates based, lipid based, nucleic acid based, natural organic based, synthetically derived organic based, antibody based and metal based substances. In preferred embodiments, the protein or antibody based substance may products of combinatorial libraries.

In yet a further embodiments, the invention relates to a system for assaying a drug for its effect on the central nervous system, said system comprising:

groups of test animals being at least one control group and an identical number of test groups;

a carrier, a general non-selective muscarinic receptor blocker in said carrier, a selective muscarinic receptor 1 (M1) blocker in said carrier and means for injecting the same into said animals;

an acetylcholinesterase (AChE) blocker and means for injecting the same into said animals;

an AChE, M1 and M2 blocker, and means for injecting the same into said animals;

means for sacrificing said animals and for dissecting their brains;

means for assessing AChE catalytic activity or AChE isoform variance, or any combination thereof in said dissected brains.

Particular reagents comprised in the system of the invention are identical with those employed in the evaluation method of the invention.

All the above and other characteristics and advantages of the invention will be further understood through the following illustrative and non-limitative description of preferred embodiments thereof, with reference to the appended drawings.

### BRIEF DESCRIPTION OF THE FIGURES

FIG. 1: Schematic of the AChE locus and transcripts.

Shown are the three different AChE splice variants, the AChE-S mRNA (S, synaptic form), the AChE-E mRNA (E, erythrocyte form) and AChE-R mRNA (R, readthrogh). Exons (1, 2, 3, 4, 5 and 6) and introns (1′ and 4′) are indicated. Nucleotide 22,465 corresponds to the ACHE transcription start site. Consensus binding sites for the noted transcription factors are represented by wedges and stars (top).

FIGS. 2A-2B: The Experimental Model

FIG. 2A: The experimental model: Rats were first injected with a mixture of propylenglycol/saline (PG/S, 1:3), scopolamine (non-selective muscarinic antagonist, 0.3 mg/Kg, i.m.) or pirenzepine (selective M1-antagonist, 2.5 mg/Kg, i.m.). 45 min later, the animals were injected with either PG/S or O,O-Diisopropyl phosphorofluoridate (DFP, AChE inhibitor, 1 mg/Kg, i.p.). Rats treated with N-octyl 3-(dimethylcarbamoyloxy) pyridinium bromide (octylpyridostigmine, PO, AChE inhibitor and partially selective M1/M2-antagonist, 20 mg/Kg, i.m.) were injected only once. Animals were sacrificed 3 or 24 h after the second injection.

FIG. 2B: Molecular structure of PO, DFP, pipenzepine and scopolamine. Abbreviations: Exp. Mod.=Experimental model; rts.scrf.+br.disct., rats sacrificed and brains dissected, T=time, hr=hour.

FIG. 3: Pre-treatment with M1 muscarinic blocker exacerbates the in vivo efficiency of AChE inhibition by DFP

Specific activity of ACHE in the somatosenory cortex of the various treated rat groups 3 and 24 hr after DIP (+) or PG/S (−) injection. Data are shown in bar graphs as a percentage of the AChE activity in the naive/non-treated group (100%). Note the significant (p<0.05) reduction of AChE activity in animals sacrificed 3 h after exposure to DFP together with the selective M1 antagonist, pirenzepine. Abbreviationa: act. Cont.=activity of control, nv=naïve.

FIGS. 4A-4B: DFP block increases the fraction of AChE monomers in a muscarinic antagonist-suppressible manner.

FIG. 4A: Representative sedimentation profiles of AChE in cortical extracts from the various treated groups as described in FIG. 2A. All sedimentation profiles were aligned with bovine catalase which served as a sedimentation marker (11.4 S, arrow). Black areas under the sedimentation curves indicate the G1 fraction.

FIG. 4B left: Representative sedimentation profiles of AChE in cortical extract from naive animals.

FIG. 4B right: G1/G4 ratios calculated based on the above sedimentation profiles. The horizontal line indicates the average G1/G4 ratio in naive animals.

Abbreviations: sedim.=sedimentation; act.=activity, rat=ratio.

FIGS. 5A-5B: DFP block diverts the stress-induced transcriptional activation in a muscarinic antagonist-suppressible manner

Shown are confocal microscopy images of the in situ hybridization rhodamine signals in the hippocampal CA3 region using cRNA probes for the AChE-S or ACHE-R mRNA. As first (1st) injection either a mixture of propylenglycol/saline (PG/S, 25:75), scopolamine (Scop 0.3 mg Kg i.m.) or pirenzepine (Pirenz., 2.5 mg/Kg i.m.) was applied. 45 min later the animals were injected (2nd) with either PG/S again (A2-A5, B2-B5) or diisopropylphosphofluoronate (DFP, 1 mg/Kg i.p., A6-A9, B6-B9). Animals were sacrificed either 3 or 24 hr following the last injection. Representative confocal images for naive mice are shown in A1 and B 1

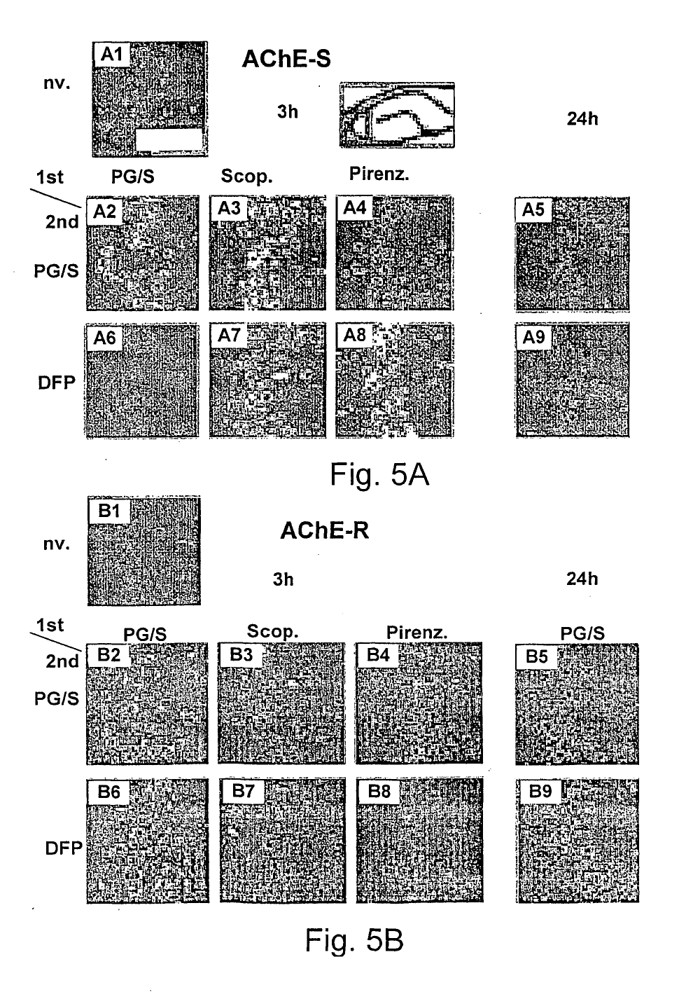

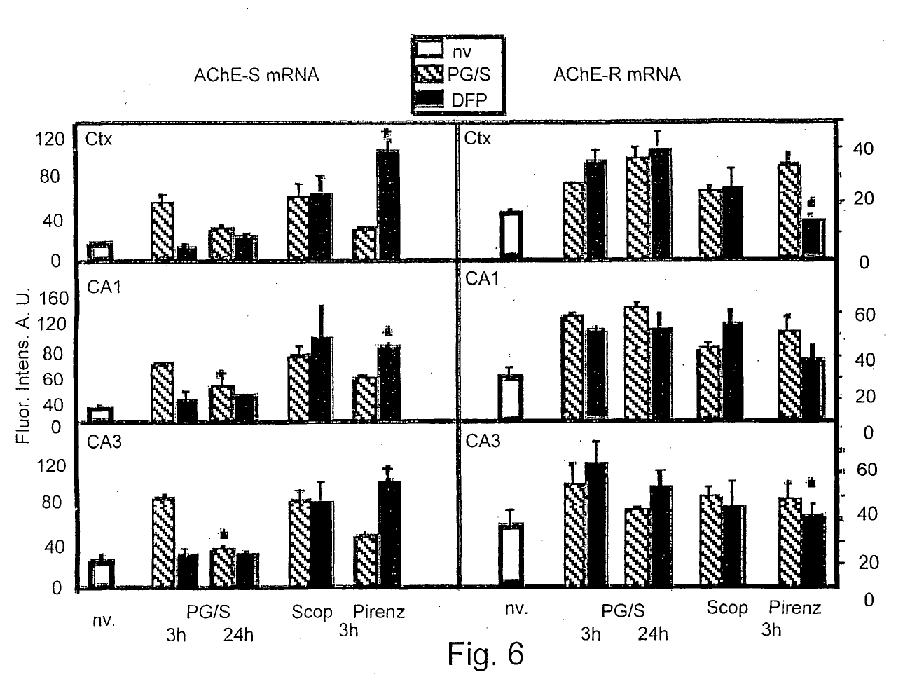

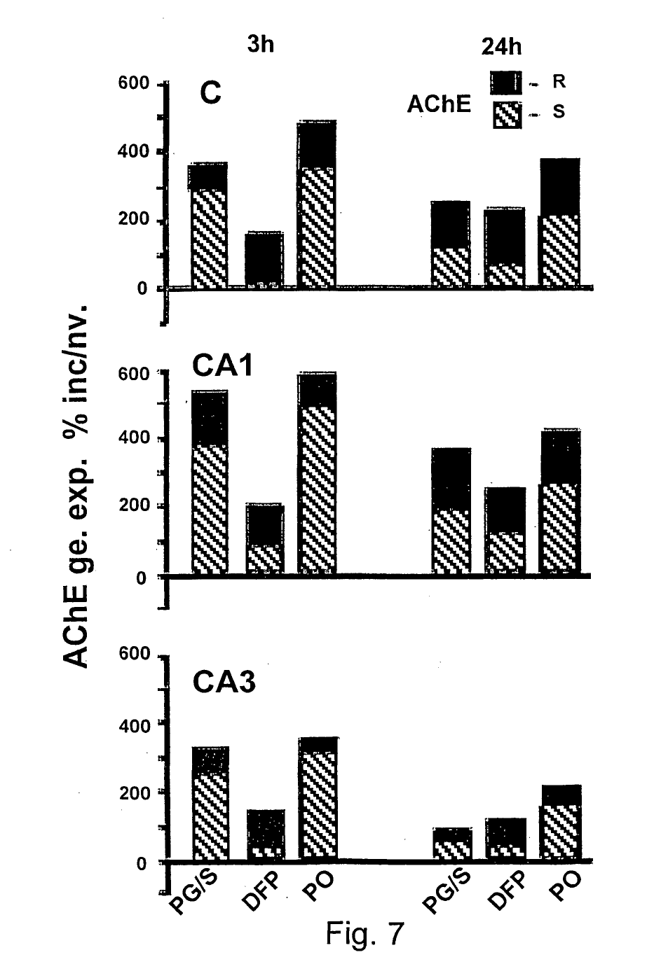

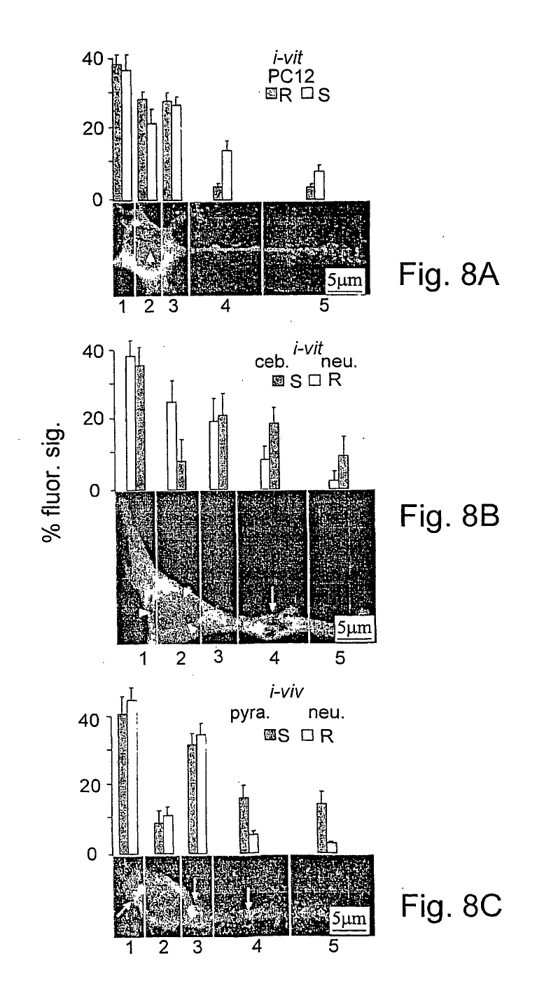

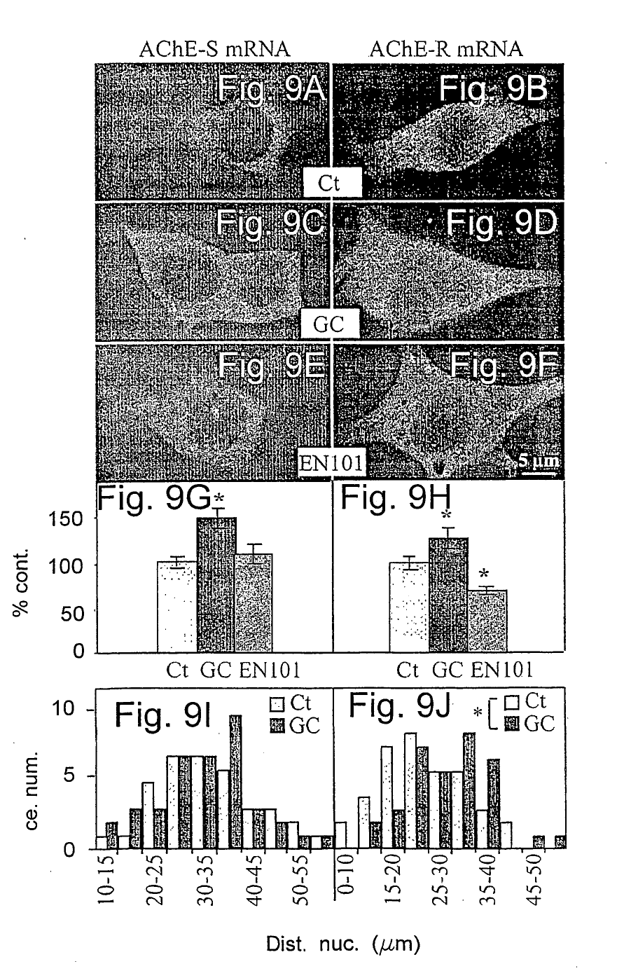

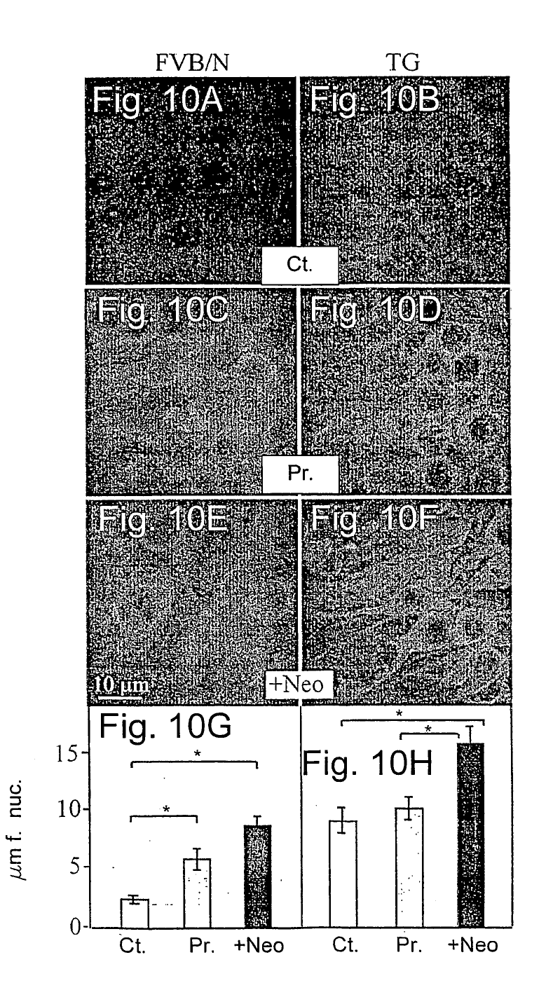

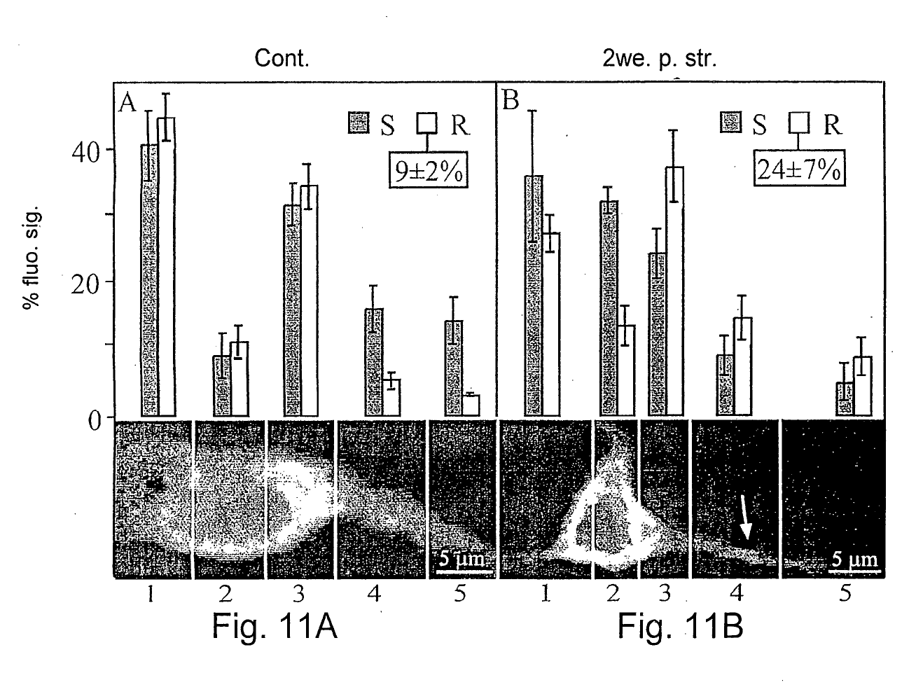

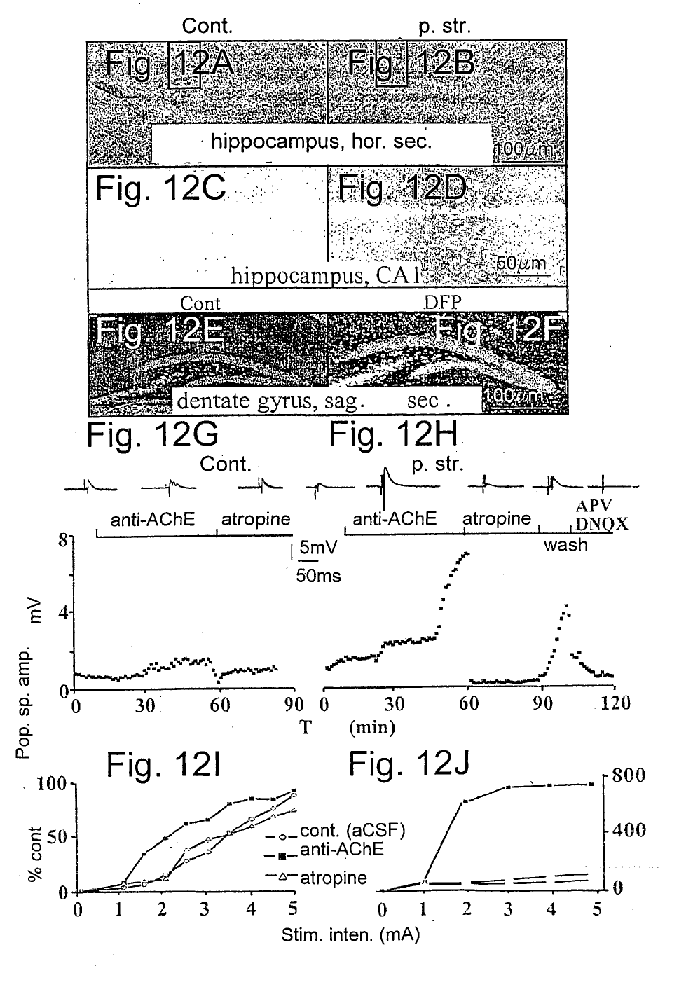

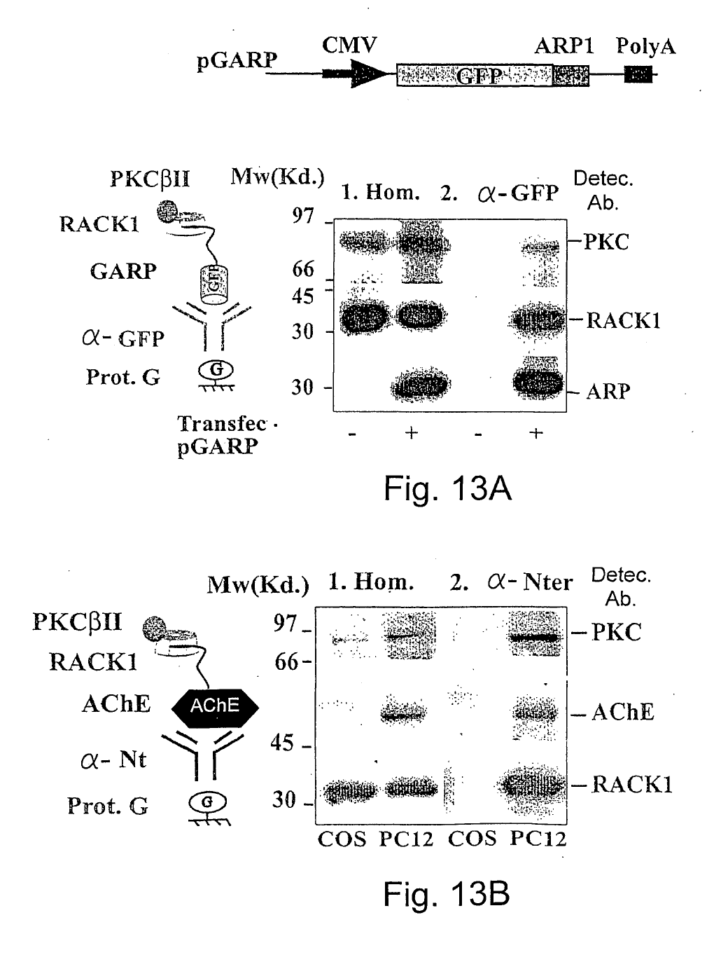

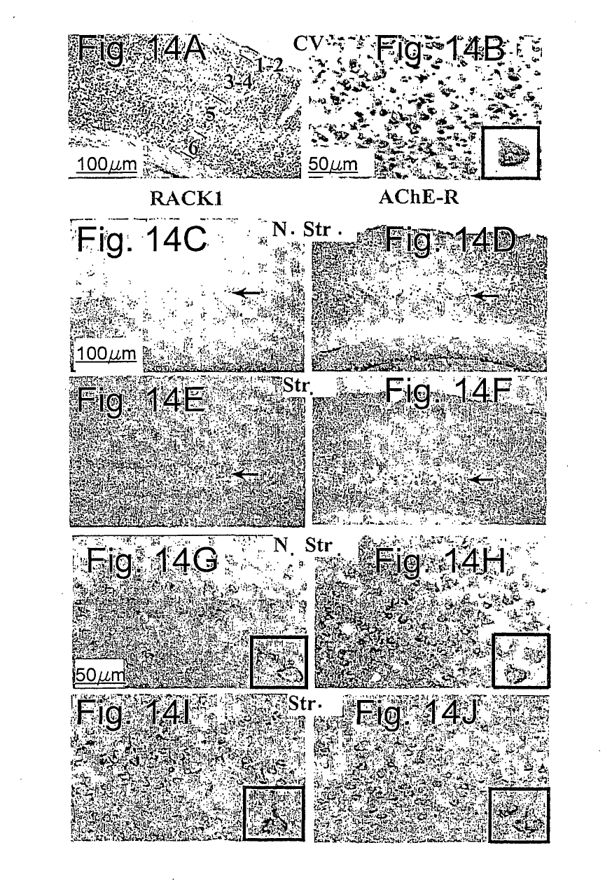

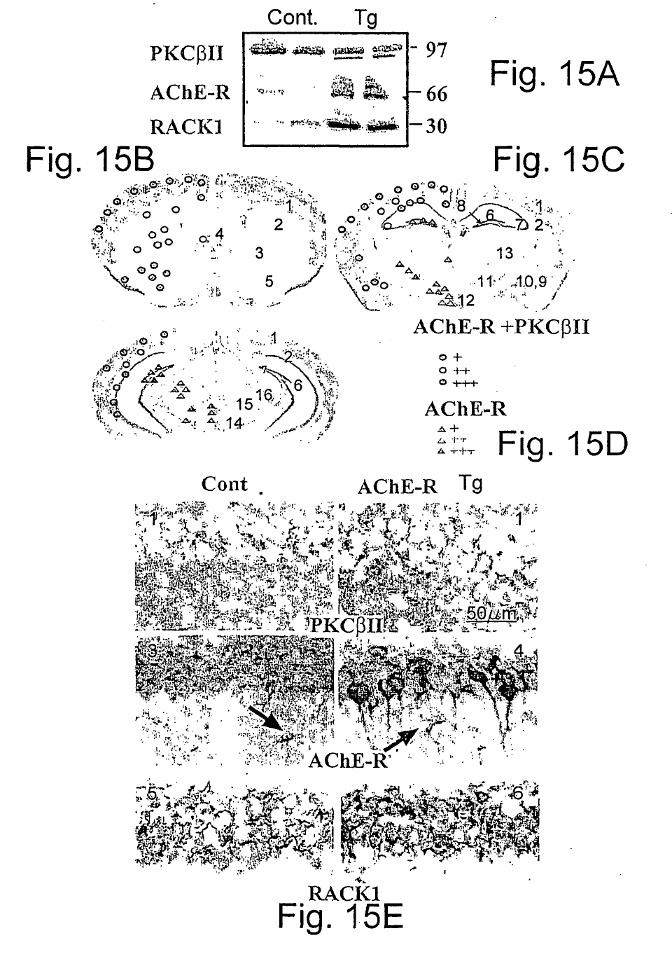

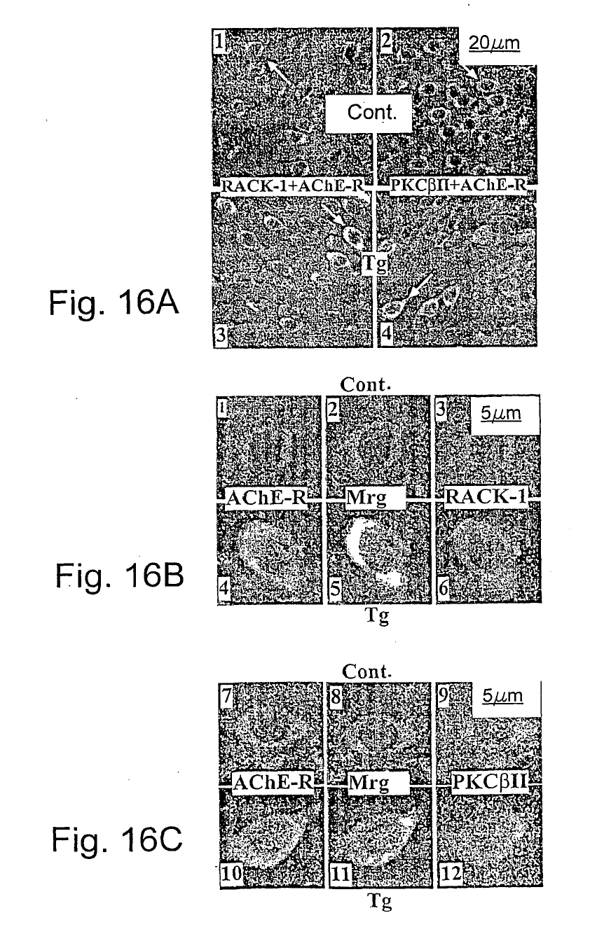

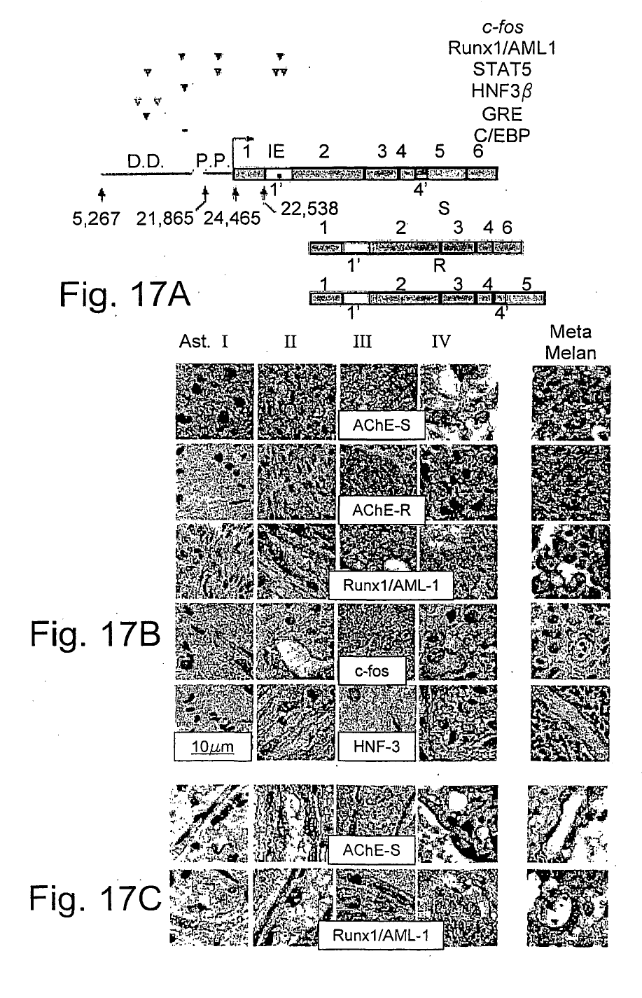

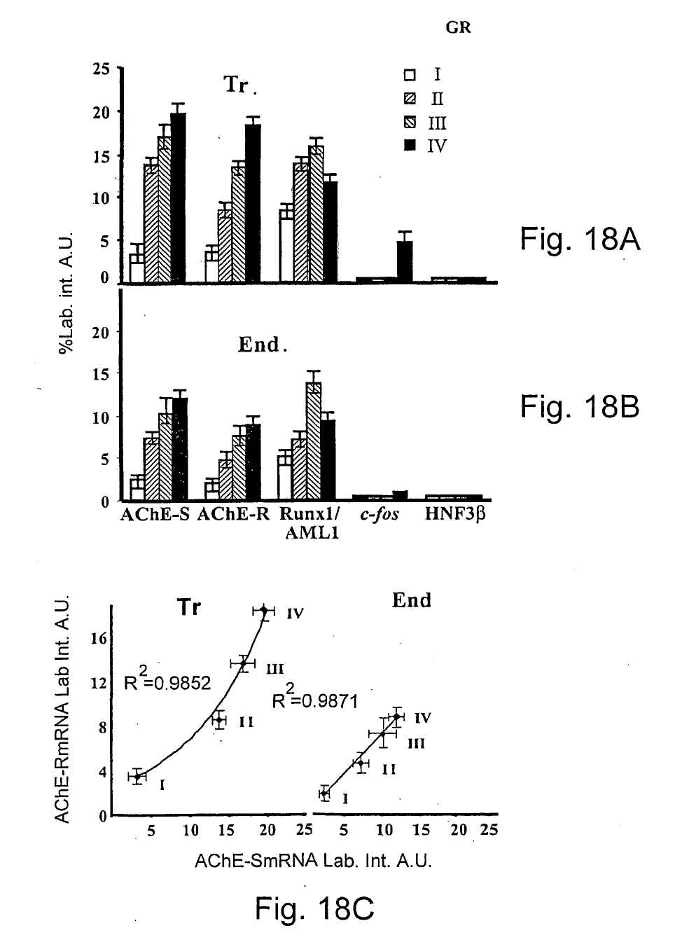

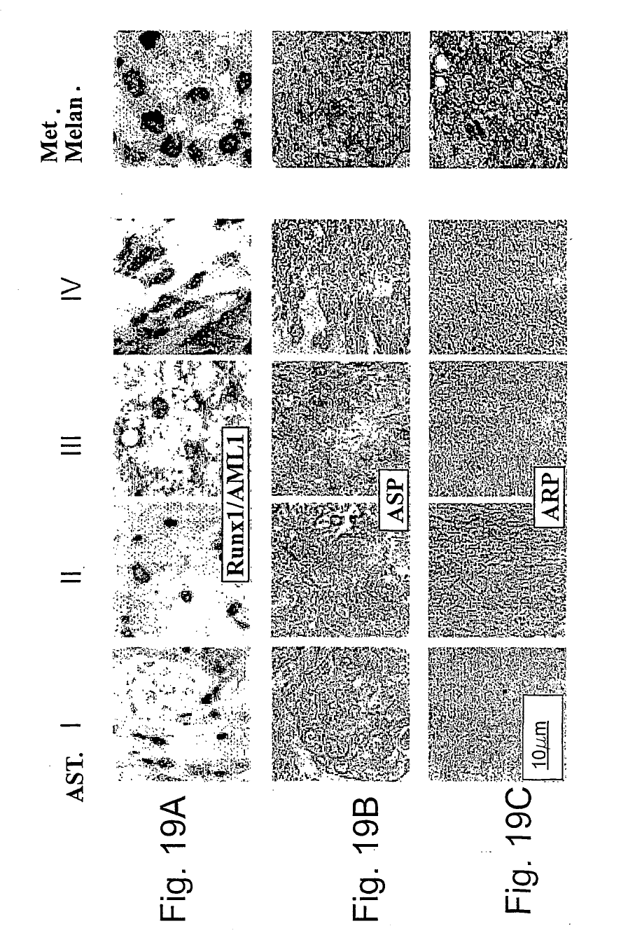

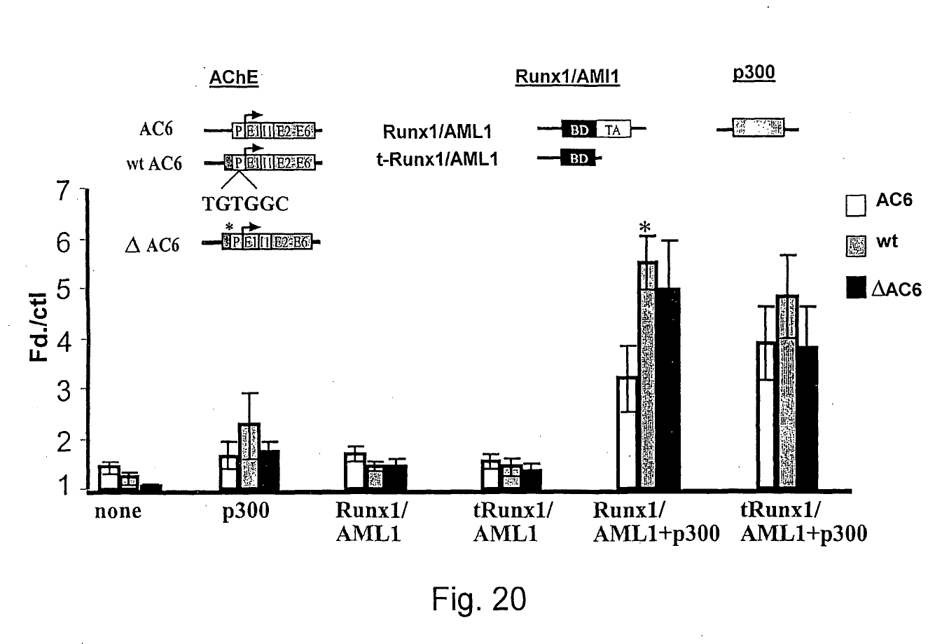

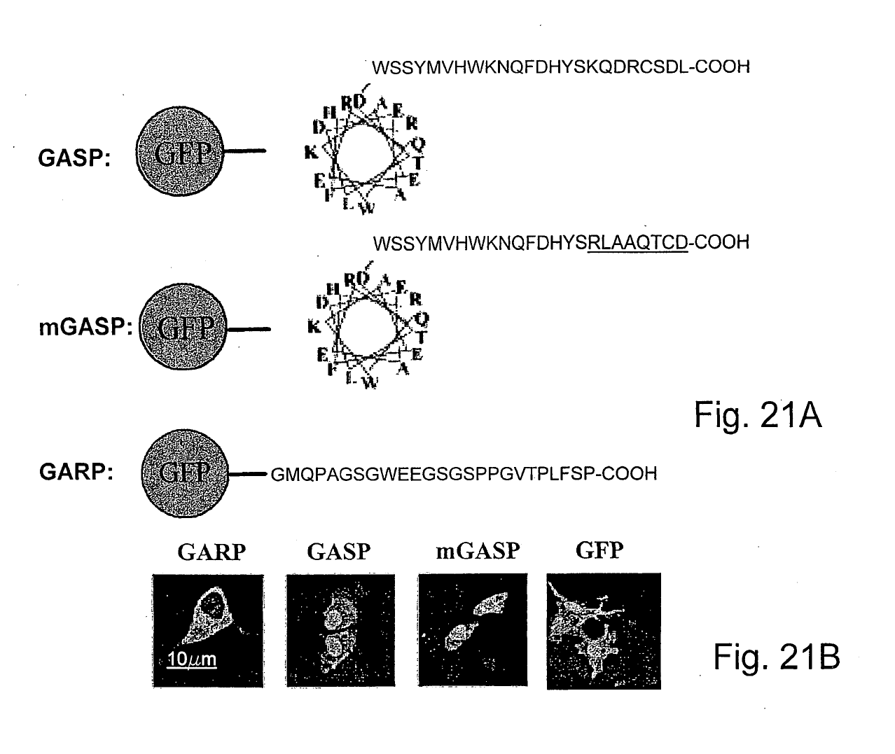

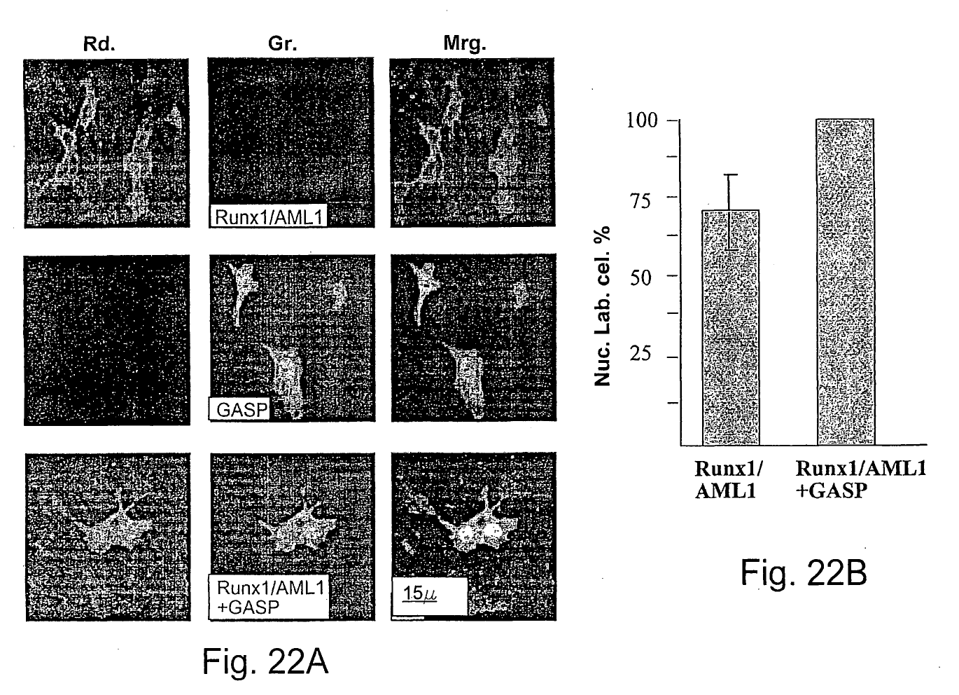

FIG. 5A: cRNA probe for AChE-S.

FIG. 5B: cRNA probe for ACHE-R.

Abbreviations: nv.=naïve, 1^{st}=first, 2^{nd}=second.

FIG. 6: Long-lasting multiregional shift in AChE alternative splicing following cholinergic insults.

Semiquantitative analysis of AChE-S and ACHE-R mRNA after in situ hybridization with rhodamine detection. The intensity of the fluorescent signal is presented in arbitrary fluorescent units for the motor cortex layer I-III as well as for the CA3 and CA1 regions of the hippocampus. Rats were injected first with either a mixture of propylenglycol/saline (PG/S, 25:75), scopolamine (Scop 0.3 mg/Kg i.m.) pirenzepine (Pirenz., 2.5 mg/Kg i.m.). Animals were injected again after 45 minutes, with either PG/S or diisopropylphosphofluoronate (DFP, 1 mg/kp i.p.). Each group included two to three rats. Statistic analysis was done with Student's t-test. Abbreviations: Fluor.intens.=Fluorescence Intensity; A.U.=arbitrary units; ctx=cortex, nv.=Naïve.

FIG. 7: AChE transcriptional activation and splicing under cholinergic insults in different brain regions.

Shown is the relative increase in AChE transcription rate height of the bars) in comparison to naive rats and the relative amounts of the two alternative splicing variants, AChE-S (white and black/white pattern) and AChE-R (black) in the overexpressed ACHE mRNA. Presented are the data for the motor cortex layer I-III as well as CA3 and CA1 region of the hippocampus. As the first injection the rats received either propylenglycol/saline (25:75), scopolamine (0.3 mg Kg i.m.) pirenzepine (2.5 mg/Kg i.m.) and 45 minutes later as second injection either propylenglycol/saline or diisopropylphosphofluoronate (1 mg/Kp i.p.). N-octyl 3-(dimethylcarbamoyloxy) pyridinium bromide (20 mg/Kg i.m.) was only injected once. Rats were sacrificed 3 or 24 h after the second injection. Abbreviations: ge exp=gene expression; inc./nv.=increase over naive; ctx=cortex; PG/S propylenglycol/saline; Scop.=scopolamine; Pirenz.=pirenzepine; DFP=diisopropylphosphofluoronate; PO=pyridinium bromide.

FIGS. 5A-8C: Variant-specific subcellular distribution of AChE mRNAs.

Shown are percentages ±SEM of FISH signals for the S and R transcripts (each totaling of 100%) in 3 parts of the perikaryon and 2 neurite areas of different cells:

FIG. 8A: PCl₂ cells.

FIG. 8B: cultured primary cerebellar neurons.

FIG. 8C: pyramidal neurons from a paraffin-embedded slice of the prefrontal cortex.

Note nuclear labeling of the R transcript in cultured cerebellar neurons (arrowheads) and the punctated pattern of transcript accumulations under in viVo conditions (arrows, C).

Abbreviations: i-vit=in vitro, i-viv=in vivo Ceb. neu.=cerebellar neurons, Pyrm. Neu.=pyramidal neurons, fluor. Sig.=fluorescent signal.

FIGS. 9A-9J: Corticosterone induction and antisense suppression of AChE mRNA in PC12 cells.

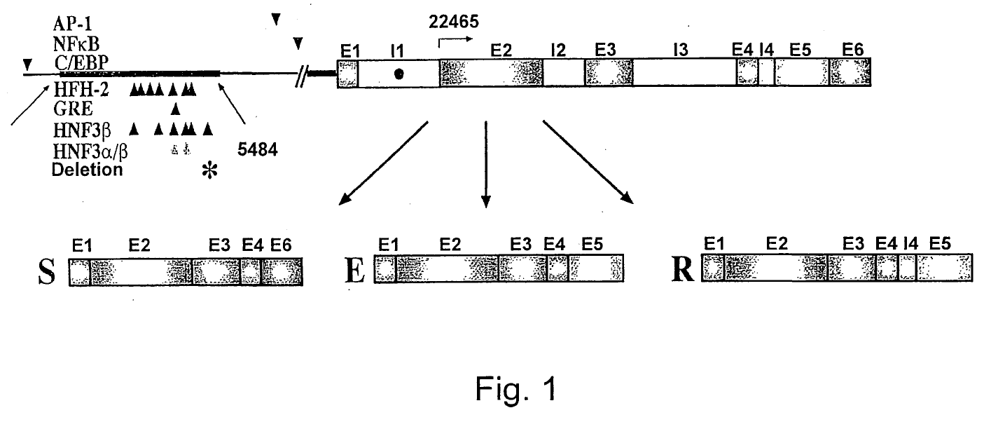

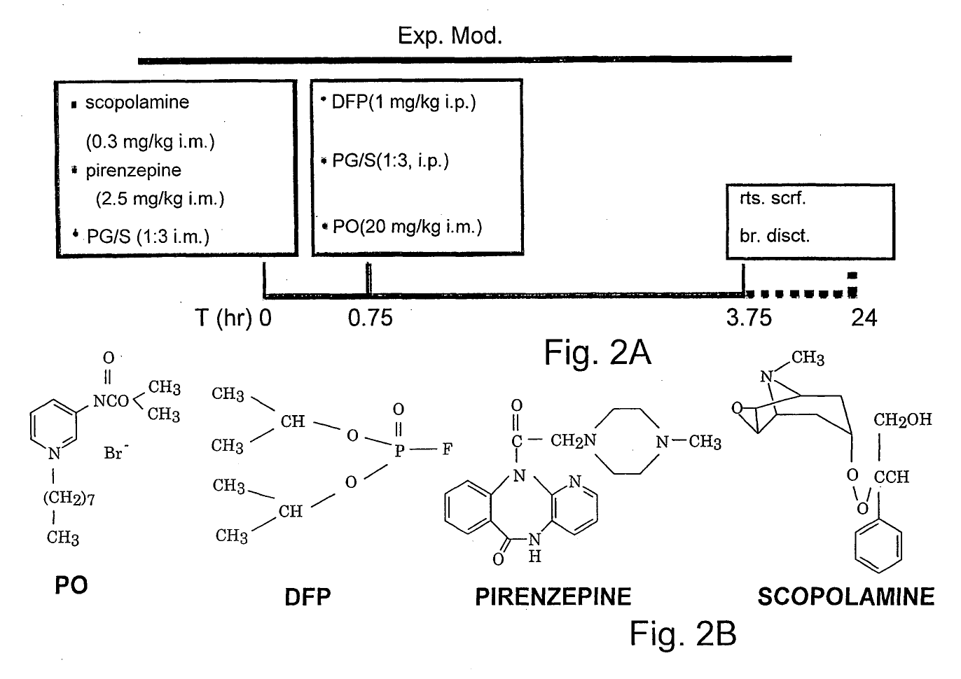

Shown are confocal micrographs (FIGS. 9A-9F), average labeling intensities (FIGS. 9G, 9H) and distributions of distances from the nucleus to the cell border (FIGS. 9I, 9J) of the S (red) and R (green) transcripts in PC12 cells (n=40) under control conditions (A, B), following 3 hrs in 10 μM corticosterone (FIGS. 9C, 9D) or 1.5 nM EN101 (FIGS. 9E, 9F). Asterisks indicate columns with significant differences from controls (p<0.05). Panels I-J present the extents of AChE-S and ACHE-R mRNA in □m from the nucleus. Abbreviations: Dis. Nuc.=distance from nucleus, ce num=cell number, % cont=% of control.

FIGS. 10A-10H: Increased dendritic translocation of hippocampal AChE-R mRNA under surgical and chemical stressors.

Confocal field images of murine hippocampal CA1 neurons from normal FVB/N mice and transgenic anmnals' overexpressing human AChE, under different conditions.

FIGS. 10A, 10B: control conditions (Ct=control, FIGS. 10A, 10B).

FIGS. 10C, 10D: 2 days following cannula implantation and pefusion with artificial cerebrospinal fluid (Pr-Probe, FIGS. 10C, 10D).

FIGS. 10E, 10F: following injection of 125 nmol neostigmine (+Neo, FIGS. 10E, 10F).

FIGS. 10G, 10H: Neuritic translocation of detectable AChE-R mRNA labeling (in μm from nucleus) was measured for 30 neurons from at least 2 animals in each group (G, H). Significant differences from noted values are starred. Abbreviations: ct.=control, Pr=probe, Neo=Neostigmine, μm f. nuc.=μm from nucleus.

FIGS. 11A-11B: In vivo neurite translocation of the R transcript following forced swim stress.

Labeling and quantification were as in FIG. 9. Mice were stressed for 4 consecutive days (2×4 min forced swim), and sacrificed 2 weeks later. Insets: AChE-R mRNA fractions in neurites.

FIG. 11A: for controls, the fraction of AChE-R in segments 4+5 was 9±2%,

FIG. 11B: for two weeks post-stress, 24±7%.

Abbreviations: % fluro. Sig. (% of fluorescent signal), 2 Wee. P. str.=2 weeks post-stress.

FIGS. 12A-12J: Hypersensitivity in hippocampal slices under long-lasting overexpression of AChE-R mRNA.

FIGS. 12A-12F: Neuronal overexpression Shown is AChE-R mRNA labeling in brains from control mice (FIGS. 12A, 12C, 12E) as compared with pre-stressed mice (FIGS. 12B, 12D) or mice pre-exposed 1 month earlier to DFP (0.1 mg/kg, 3 consecutive daily injections, FIG. 12F).

FIGS. 12G-12J: Cholinergic hypersensitivity Shown are extracellular recordings in the CA1 area in response to stratum oriens stimulation of hippocampal slices from controls (FIGS. 12G, 12I) or 1 month following consecutive daily stress sessions (FIGS. 12H, 12J). (The following concentrations were applied: anti-AChE physostigmine, 10 □M; atropine, 10 μM; APV, 50 μM; DNQX, 20 μM. Abbreviations: cont.=control, p. str.=post stress, hor. Sec.=horizontal sections, sag. Sec.=saggital section stim. Inten.=stimulus intensity, popu. sp. amp.=population spike amplitude, T=time, min=minutes.

FIGS. 13A-13B: Immunoprecipitation of AChE-R/RACK1/PKCβII complexes

FIG. 13A: Top: CMV-based vector encoding pGARP, a GFP fusion protein with ARP1. Bottom: The drawing on the side represents the experimental concept.

1. Homogenates: Shown are immunolabeled RACK1 and PKCβII (but not ARP1) in non-transfected COS cell homogenates (−). In the presence of transfected GARP (+), COS cells show a band in the position correspondent to ARP.

2. Anti-GFP: Inimunoprecipitation with anti-GFP antibodies precipitates PKCβII, ARP and RACK1 in PGARP transfected but not in non-transfected COS cells.

FIG. 13B: RACK1 and PKCβII were co-immunoprecipitate in cell lysates prepared from AChE-R expressing cells (PC12) using anti-AChE antibodies. Precipitates were separated on SDS PAGE and gels were bloted. Membrane protein blots were subjected to various labeling experiments using anti PKC, RACK1 and AChE antibodies. Schematic on the left represents the experimental concept:

1. Homogenates (Hom): PKCβII and RACK1 are immunodetected in homogenates of COS cells, which do not express AChE, and PKCβII, RACK1 and AChE are detected in PC12 cell homogenates.

2. Anti-AChE N-terminus (N-term): Dissolved immunoprecipitation complexes created with antibodies to the N-terminus of AChE display no signals in COS cells, but are positive for all three partner proteins in PC12 cells, demonstrating AChE requirement for the creation of these complexes. Abbreviations: Prot. G=protein G sepharose, Ab.=antibody, Transfec.=transfection; Detec. Ab.=detection antibody, Hom=homogenate, α=anti, Nt=N-terminal.

FIGS. 14A-14J: RACK1 and AChE-R co-overexpression in parietal cortex and CA1 neurons under stress

Shown are parietal cortex sections stained with cresyl violet or with anti-RACK1 or anti-AChE-R antibodies, in lower and higher magnifications. Note uneven labeling patterns of both proteins in the cytoplasm and proximal processes of individual pyramidal neurons in the parietal cortex and hippocampus CA1 (insets). Note RACK1 and AChE-R expression increases in layers 5 (arrows) of the parietal cortex under stress.

FIG. 14A: Cresyl violet staining, lower magnification.

FIG. 14B: Cresyl violet staining, higher magnification.

FIG. 14C: No stress, anti-RACK1 staining, lower magnification.

FIG. 14D: No stress, anti ACHE-R staining, lower magnification.

FIG. 14E: Stress, anti-RACK1 staining, lower magnification.

FIG. 14F: Stress, anti-AChE-R staining, lower magnification.

FIG. 14G: No stress, anti-RACK1 staining, higher magnification.

FIG. 14H: No stress, anti ACHE-R staining, higher magnification.

FIG. 14I: Stress, anti-RACK1 staining, higher magnification.

FIG. 14J: Stress, anti-AChE-R staining, higher magnification.

Abbreviations: N. Str.=no stress; Str.=stress.

FIGS. 15A-15E: Transgenic AChE-R overexpression intensifies neuronal RACK1 and PKCβII labeling in hippocampal CA1 neurons.

FIG. 15A: Immunoblot analysis. The immunoblot shows the bands corresponding to PKCβII, AChE-R and RACK1 following gel electrophoresis of clear hippocampal homogenates from two FVB/N controls and two sex and age-matched AChE-R transgenic mice (Tg). Note the intensified staining in transgenics and the fast migrating additional PKCβII band, which could not be detected in controls. Representative results from five reproducible experiments.

FIGS. 15B-D: Partial overlaps in neuronal AChE-R accumulation and PKCβII distributions.

Shown are selected brain sections (posterior to Bregma 0.0-0.2 mm, 1.5-1.7 mm and 2.9-3.1 mm respectively) and the corresponding subregions where AChE-R accumulation (triangles) or PKCβII co-labeling in AChE-R accumulating neurons (circles) were detected. Staining intensity was low (+), medium (++) or high (+++). The corresponding subregions are numbered as follows: 1, Cortex upper layers; 2, Cortex lower layers; 3, striatum; 4, lateral septum; 5, piriform cortex; 6, hippocampus CA1; 7, hippocampus CA3; 8, hippocampus dentate gyrus; 9, basolateral amygdala; 10, central amygdala; 11, lateral hypothalamus; 12, ventromedial hypothalamus; 13, ventral lateral thalamus; 14, Edinger-Westphal nucleus; 15, Red nucleus; 16, Pre-tectal area.

FIG. 15E: Hippocampal immuohistochemistry. Shown are parallel CA1 regions from representative control and AChE-R transgenics stained with antibodies toward PKCβII, AChE-R or RACK1, as indicated. Note the intensified non-homogeneous staining of hippocampal neurons in the brain of transgenics for both AChE-R and RACK1, the relatively high background staining of PKCβII and the microglia (arrows) positive for ACHE-R. Abbreviations: Cont.=control; Tg.=transgenic.

FIGS. 16A-16C: Double-labeling highlights the AChE-R modulation of AChE-R/RACK1/PKCβII complexes.

FIG. 16A: PKC activity is increased in ACHE-R transgenics PKC activity in brain homogenates from AChE-R transgenic was measured using the PKC assay kit (Upstate Biotechnology). Note the elevation of PKC activity in the different brain regions in the AChE-R transgenics as compered to FVB/N controls.

FIGS. 16B-C: Shown are merged confocal micrographs from individual upper layer parietal cortex neurons of FVB/N and AChE-R overexpressing transgenic mice, co-immunolabeled with AChE-R/RACK1 or AChE-R/PKCβII. Staining was with antibodies to ACHE-R (green) and RACK1 or PKCβII (red); merged micrographs show yellow signals for overlap staining, with orange regions reflecting high partner levels. Note the uneven, distinct distributions of the analyzed antigens in cortical neurons, with AChE-R labeling demonstrating perikaryal distribution, RACK1 more mobilized toward the perikaryal region identified in top sections (No. B5) and PKCβII highlighted in dense clusters co-localized with both RACK1 and AChE-R (No. C11).

FIG. 16B: co-immunolabeling with ACHE-R and RACK1.

FIG. 16C: co-immunolabeling with AChE-R and PKCβII.

Abbreviations: Cont.=control; Mrg.=merge, Tg=transgenic.

FIGS. 17A-17C: Expression of AChE variants, Runx1/AML-1, c-fos and HNF-3β in astrocytoma and metastatic brain melanoma.

FIG. 17A: Schematic of the AChE locus and transcripts.

Depicted is the reverse sequence of the cosmid insert (Genebank Accession No. AF002993) containing the ACHE gene and 22 kb of its upstream sequence. Exons (1, 2, 3, 4, 5 and 6) and introns (1′ and 4′) are indicated. Arrows designate starting positions of the distal enhancer domain (DD), proximal promoter (PP) and the intronic enhancer (IE) along the cosmid reverse sequence. Nucleotide 22,465 corresponds to the ACHE transcription start site. Consensus binding sites for the noted transcription factors are represented by wedges and stars (top).

FIG. 17B: Results of in situ hybridization using 5′-biotinylated cRNA probes selective for the AChE-S and AChE-R mRNA variants as well as for the Runx1/AML1, c-fos and HNF3β mRNA, on sections of human astrocytoma at various pathological grades and in metastatic brain melanoma. Abbreviations: Met.Melan., Metastatic Melanoma.

FIG. 17C: Expression patterns in endothelial vascular cells supplying astrocytoma and metastatic brain melanoma. Results of in situ hybridization performed in tumor tissue sections including vasculature endothelium. Experimental details as in B.

FIGS. 18A-18C: Expression patterns of AChE variants and Runx1/AML1 in astrocytoma tumors correlate.

Graphs represent mRNA labeling intensity mean values ±SEM of the percentage of red pixels, falling within a defined intensity range.

FIG. 18A: Malignant tumor cells.

FIG. 18B: Vascular endothelial cells surrounding the tumor tissue.

FIG. 18C: Mean values (+SEM) of AChE-R mRNA plotted as a function of AChE-S mRNA labeling, in tumor cells (left) and in vascular endothelial cells (right) of astrocytoma of grades I-IV. R² values for best fit curves were calculated in each case.

Abbreviations: Gr, grade; Tr, tumor; End, endothelial; Lab. Int., labelling intensity; A.U., arbitrary units.

FIGS. 19A-19C: Runx1/AML1 and AChE variants detection in astrocytoma and metastatic brain melanoma.

Immunomicrographs of graded human astrocytoma and brain metastatic melanoma labeled with antibodies that recognize

FIG. 19A: Runxl/AML1,

FIG. 19B: AChE-S C-terminal peptide (ASP), and

FIG. 19C: AChE-R C-terminal peptide (ARP).

Abbreviations: Ast, astrocytoma; Met.Melan., Metastatic Melanoma.

FIG. 20: Runx1/AML1-p300 co-transfection facilitates AChE expression in COS1 cells.

Graph showing AChE activity in COS1 cells transfected with construct AC6 (open bars), wtAC6 (gray bars) or ΔAC6 (black bars), alone or in combination with p300, Runx1/AML1, tRunx1/AML1, Runx1/AML1 and p300, or t-Runx1/AML1 and p300.

*=statistically significant increase, when compared to AC6.

Inset: AChE and Runx1/AML1 expression constructs used for transfection experiments. AChE distal enhancer domain, normal and mutated fragments are shown as dark gray boxes; mutated sequence marked with a star, the proximal promoter (P) harbors a Runx1/AML1 DNA consensus binding site, followed by intron 1 (I1) and numbered exons (E). Runx1/AML1 includes the binding domain (BD) and the trans-activating domain (TA), t-Runx1/AML1 encodes a truncated Runx1/AML1, lacking the transactivation domain.

Abbreviations: fd./ctl, fold over control.

FIGS. 21A-21B: AChE C-terminal peptides target distinct sub-cellular sites.

FIG. 21A: Shown are GFP-AChE fused expression constructs. GFP-ASP (GASP) encods a fusion protein of GFP and the C-terminal peptide of humanAChE-S, mutated (m)GASP encodes GASP with replaced last 8 amino acids (boxed letters) and GARP encodes a GFP fusion with the “readthrough” ARP. Hydrophobic residues are marked red.

FIG. 21B: Representative COS1 cells transfected with the above vectors.

FIGS. 22A-22B: GASP augments Runx1/AML1 nuclear localization in transfected Cos1 cells.

FIG. 22A: Shown are representative confocal micrographs of Cos1 cells transfected with the noted vectors. Green fluorescence depicts the GFP fusion protein and red Runx1/AML1.

FIG. 22B: Columns show precentage ±SEM of nuclear-labeled Runx1/AML1 COS 1 cells transfected with the noted expression vectors.

Abbreviations: Rd, red; Gr, green; Mrg, merge; nuc.lab.cel, nuclear labeled cells.

### DETAILED DESCRIPTION OF THE INVENTION

For the purposes of clarity, the following terms are defined herein:

ACh: Acetylcholine;

ACHE: Acetylcholinesterase;

AChE-R: Readthrough isoform of AChE;

ACHE-S: Synaptic isoform of AChE;

anti-AChE: drug or substance that inhibits ACHE activity;

ARP: ACHE “readthrough” peptide;

ASP: AChE “synaptic” peptide;

GARP: chimeric fusion protein between GFP (green fluorescent protein) and ARP;

GASP: chimeric fusion protein between GFP (green fluorescent protein) and ASP;

MBP: maltose-binding protein;

PKC: protein kinase C;

RACK: receptor of activator kinase C;

Tg: transgenic;

WD domain: domain found in WD40 family proteins, which are characterized by having multiple tandem copies of a sequence of about 40 amino acids with a conserved WD pair towards the C-terminus;

AD: Alzheimer's disease;

CNS: central nervous system.

The present invention relates to a method for evaluating the effects of certain drugs on the nervous system. More specifically, the drug is evaluated vis-à-vis its influence on the expression and function of AChE and its variants, as described herein.

To determine whether well-balanced cholinergic neurotransmission is a key prerequisite for elevating AChE gene expression and shifting the splicing of AChE mRNA under stress, to compare the consequences of stress to those of anti-AChE exposure, and to search for the relevant receptors, the inventors combined molecular and cell biology with biochemistry approaches. The picture emerging from these combined approaches was that of a more global reading of AChE response, alongside the upstream and downstream pathways involved in this response. This is a key point for a better understanding of the cholinergic system, particularly with regards to the development of new drugs directed towards diseases or conditions affecting this system, as well as the nervous system in general, as well as detecting adverse effects of any candidate drug on the nervous system, particularly the CNS.

The first aspect of the present invention relates to a method for evaluating an effect on the nervous system of a test drug. Once a drug undergoes testing through this method, a profile is generated, which reflects its global effect on the cholinergic system.

Thus, the invention relates to a method and system for evaluating an effect on the nervous system of a test drug by comparing the effect of such drug on AChE catalytic activity or isoform variance in the brain of a test animal following challenge by an AChE blocker (e.g. DFP) or a blocker of AChE and muscarinic receptors M1 and M2 (e.g. pyridostigmine) and comparing this effect with that of a known agent, preferably a non-selective muscarinic receptor blocker (e.g. scopolamine) or a M1 receptor blocker (e.g. pirenzepine).

More specifically, the invention relates to a method comprising of the following steps:

In a first step, the following groups of test animals are provided: (i) at least one control group, and preferably three control groups, to be pre-treated by a known agent selected from a carrier or vehicle, which is identical to the one in which the test drug is dissolved in, or a general non-selective muscarinic receptor blocker or a selective muscarinic receptor 1 (M1) blocker; and (ii) an identical number of corresponding test group, to be pre-treated with the test drug carried in said vehicle. Optionally, a further kind of test groups may be provided, namely control groups of naive test animal, that are not pre-treated.

In step two, the animals in the control group/s are injected at time zero [0] with an agent selected from a carrier or vehicle, as defined above, a general non-selective muscarinic receptor blocker, and a selective muscarinic receptor 1 (M1) blocker, and at the same time each of the animals of the test group/s (ii) is injected with the test drug.

In the third step, after at least about 45 minutes and not more than about 90 minutes, preferably between 45 minutes and 1 hour of the first pre-treatment injection, the animals of group (i) are injected with either a carrier, in case a carrier or a general non-selective muscarinic receptor blocker or a M1 blocker was used in the previous step; or an AChE blocker, in case a carrier or a general non-selective muscarinic receptor blocker or a M1 blocker was used in the previous step; or an AChE, M1 and M2 blocker, in case a carrier was used in the previous step. At the same time, the animals of the test group/s (ii) are injected with the same agent used for the corresponding test group (ii).

In the next step, after at least 2 hours and not more than 4 hours, preferably 3 hours after the second injection, a number of animals from each of the control and test group/, and the optional naïve, non-pre-treated animals group are sacrificed and their brains are dissected.

Then, after at least 24 hours and not more than 36 hours, preferably between 24 and 30 hours from the second injection, all of the remaining animals are sacrificed and their brains are dissected.

The dissected brains are then subjected to at least one analytical procedure for assessing AChE catalytic activity or isoform variance, or any combination thereof, a drug profile is generated from the results of these analytical procedures for each of the groups. Then, the profile of the test group/s is compared to the control profile/s, and the results interpreted, as will be described in more detail hereafter.

An example of a carriers (vehicle) is propyleneglycol/saline, or any other physiologically compatible solvent, carrier or diluent, which will dissolve the active agent employed, but would not affect its activity.

Examples of general non-selective muscarinic receptor blockers include, but are not limited to scopolamine and atropine.

An example of selective M1 blocker is pirenzepine, but other such blockers are suitable.

An examples of AChE blocker is diisopropylfluorophosphonate (DFP), but many other AChE inhibitors are available.

An example of a “total” blocker, which blocks AChE, M1 and M2 is octylpyridostigmine (PO), but other such blockers are available.

As described in Example 1, simultaneous analysis of the catalytic AChE activity, sedimentation profile and AChE mRNA expression revealed that both mild pain (the injection of the carrier) and the organophosphate AChE inhibitor DFP, induce massive transcriptional activation of the rat ACHE gene, which lasted at least 24 hours, and was common to the cortex and hippocampus. Most importantly, this response was dependent on the integrity of muscarinic neurotransmission. The increased ACh concentration following AChE inhibition induced a shift in the alternative splicing of AChE mRNA transcripts from ACHE-S to ACHE-R, primarily through activation of M1 muscarinic receptors, and yielded significant increases in the fraction of AChE monomers G1, presumably AChE-R. Interestingly, this shift was prevented by pre-treatment with M1 antagonists and even reversed under the compound effect of M1/M2 muscarinic blockade and AChE inhibition, induced by PO.

Several steps along the pathway for regulating gene expression should be activated in concert in order to initiate the above-described drug response. In the healthy brain, with well-functioning muscarinic neurotransmission, both AChE inhibitors and psychological stress elevate ACh levels at cholinergic synapses [Kaufer, D. et al. (1999) The Neuroscientist 5:173-183]. Such elevation activates muscarinic receptors, particularly the M1 excitatory post-synaptic receptor which changes the phosphorylation properties of PKC at the acceptor neurons [Bouron A and Reuter H. (1997) Proc Natl Acad Sci USA 94(22):12224-9]. Another change occurs in the M2 inhibitory pre-synaptic receptors, the activation of which in the presence of high synaptic ACh levels blocks subsequent release of ACh into the synaptic microenvironment. The experiments (Example 1) herein described demonstrate efficient maintenance of the ACHE transcriptional enhancement under PO. This may reflect synergistic contributions of the anti-AChE property of PO and its M1/M2 blockade capacity, which prevents pre-synaptic changes under increased ACh concentrations in the cholinergic synapse. Pre-synaptic cholinergic neurons are especially vulnerable in Alzheimer's disease (AD), suggesting that the effects observed under PO may be closer to those of patients under AChE inhibitors: longer duration of the transcriptional enhancement and limited shift in the alternative splicing of AChE variants, that could result in detrimental accumulation of AChE-S rather than AChE-R.

It is interesting to note that AChE-R mRNA levels in the brain samples analyzed in Example 1 were extensively elevated in rats exposed to DFP, significantly more than in animals exposed to stress alone, induced by the carrier. Addition of pirenzepine significantly decreased the levels of AChE-R mRNA, emphasizing the essential role of the M1 receptor in this process. A less prominent decrease was observed with scopolamine treatment, suggesting that impaired control over muscarinic neurotransmission would be detrimental to the feedback response to anti-AChEs, even when it is non-specific. After 24 hours, AChE-R mRNA was still overexpressed in all treated animal brains. Since the half-life of AChE-RmRNA is considerably shorter than that of AChE-S mRNA [Chan et al., (1998) supra.], which reflects a continuously excessive transcription from the AChE gene, coupled with shifted alternative splicing, under any imbalance in cholinergic neurotransmission. Both scopolamine and pirenzepine treatments did not show any effect on AChE-S or -R levels after 24 hours, most likely due to their short biological half-life.

Anti-AChEs include commonly used insecticides, prophylactic and therapeutic drugs (e.g. for the treatment of AD or myasthenia gravis) and chemical and terrorism warfare agents. The findings herein described expose previously overlooked consequences of the effects of such agents, especially in individuals with compromised muscarinic neurotransmission. Importantly, since AChE comprises not one, but three proteins, its variant composition may alter physiological processes in ways that are not yet recognizable or predictable.

The profile resulting from the analytical procedures performed in the brain samples is important for determining the effect of the test drug on the cholinergic system, whereby firstly, increased expression in the brain of AChE mRNA transcripts, with or without a shift in the alternative splicing of said transcripts from the plimary S to the normally rare R variant, indicates a feedback response to said drug and specifies its nature; secondly, an increase with time in the catalytic activity of brain AChE, with or without an increase in the ratio of globular G1 AChE monomers to G4 tetramers reflects the outcome of such feedback at the protein level; and thirdly, any diversion from the hereby characterized brain response to the irreversible ACHE inhibitor DFP reflects an impairment in muscarinic neurotransmission as a result of exposure to the tested drug.

In one embodiment, the three control groups which are preferably employed in the method of the invention are defined according to the substance injected at time zero [0]. The three control groups are therefore defined as follows: group (i-a), wherein each of the animals was injected with a carrier, as defined above; group (i-b), wherein each of the animals was injected with a general non-selective muscarinic receptor blocker, as defined above; and group (i-c), wherein each of the animals was injected with a M1 blocker, as defined above. Each control group has a corresponding test group, the animals in which are injected, after the pre-treatment, with the same agent injected to the control group.

In another embodiment, the analytical procedures utilized in the method of the invention are selected from the group consisting of determining AChE activity, determining AChE-S and/or -R isoforms ratio and determining AChE-S and/or -R mRNA variants tissue expression and distribution

The AChE isoform ratio may evaluated by any available method, for example by means of a sucrose gradient.

In another specific embodiment, the ACHE variant or isoform expression in tissues may be determined by anyone of in situ hybridization utilizing variant specific probes, and immunohistochemistry utilizing isoform specific antibodies. The determination of AChE variant or isoform expression may be measured by various means, including enzymatic, colorimetric and fluorimetric assays.

The test drug to be subjected to the method of the invention is preferably selected from the group of drugs for the treatment of conditions defined as anxiety, post-traumatic stress, Alzheimer's disease, muscle malfunctioning, neurodegenerative disorders, exposure to xenobiotics, panic, neuromuscular disorders, Parkinson's disease, Huntington's chorea, muscle fatigue, multiple chemical sensitivity, autism, multiple sclerosis and Shorgren's disease. Nonetheless, it is to be appreciated that the method of the invention is suitable for assessing the effects on the nervous system, particularly the CNS, of any candidate drug, and thus may be important in processes of regulating use of novel drugs.

“Animal” for purposes of test animal refers to any animal classified as a mammal suitable for use as an experimental model, such as rats, mice, guinea-pigs, hamsters, rabbits, dogs, cats, etc. Preferably, the mammal is a rat. It is however to be appreciated that the method and system of the invention may be adapted also for non-mammalian animals.

The transcriptional activation under the mild pain stress caused by PG/S injection may reflect the functional involvement in the distal ACHE enhancer of binding elements for the glucocorticoid receptor GRE [Grisaru D. et al. (2001) Mol Med 7:93-105]. In this context, the accumulation of brain G1 AChE monomers as described in Example 1 of the present invention is compatible with the AChE-R mRNA increases in hematopoietic cells under stress. Nitsch and coworkers (1998) [Nitsch, R. M. et al. (1998) J Physiol Paris 92(3-4):257-64] have demonstrated in cell transfection experiments M1 muscarinic receptor activation of the EGFR and AP1 sites that are found in the proximal ACHE promoter, providing a mechanistic explanation to the stress and inhibitor-induced transcriptional activation. In light of the experiments described herein, it becomes transparent that in the mammalian brain, this part of the drug response cannot take place under M1 muscarinic malfunctioning, but would be particularly efficient under M2 muscarinic inhibition.

The increasing over-expression of ACHE variants in increasingly aggressive astrocytomas, shown in Example 4, in conjunction with previous reports on the morphogenetic and proliferative activities of AChE, suggests a role for AChE in astrocytic tumor development and/or aggressiveness.

In order to evaluate astrocyte oncogenesis potential of the test drug, a specific embodiment of the generation of the drug profile further comprises the steps of: (a) treating an animal with said test drug; (b) dissecting the brain of said animal; (c) contacting said dissected brain with detectable means for determining the expression of the transcriptional activator Runx1/AML; and (d) comparing the level of expression of Runx1/AML in said brain with the level of expression of the same gene in a brain tumor sample.

As described in Example 4, the concerted increase of the transcription factor Runx1/AML1, previously not suspected to participate in astrocytic gene expression, together with the AChE variants, suggested that Runx1/AML1 upregulates AChE expression in astrocytic tumors and metastatic brain melanoma. In contrast, neither c-fos nor HNF3□, transcription factors known to affect neuronal AChE expression under various stresses, were expressed in astrocytic tumors, emphasizing the specificity of this response. The present observation that Runx1/AML1 is present in human glial cells and neurons is consistent with the expression of its homologues in Drosophila neuroblasts [Duffy, J. et al. Development 113:1223-30 (1991)], and in zebrafish olfactory placodes and in cells attached to the otic vesicle [Kataoka, H. et al. Mech Dev 98:139-43 (2000)], possibly reflecting a yet unknown role for this transcription factor in the mammalian nervous system. In the in vitro model herein described, the C-terminal peptide of AChE-S directed GFP into cultured cell nuclei and enhanced the nuclear targeting of Runx1/AML1, pointing at a corroborating mechanism whereby AChE-S assists the nuclear targeting of Runx1/AML1, which in turn, enhances AChE gene expression.

Furthermore, Runx1/AML1, together with its co-activator p300, further enhanced AChE gene expression in transfected COS1 cells. Being a weak effector of transcription by itself, Runx1/AML1 dependence on p300 re-enforces its significance. Moreover, p300 effect was increased in the presence of the AChE distal enhancer domain, suggesting a potential involvement of this region in the transcriptional regulation of AChE by Runx1/AML1. p300 may thus enhance AChE gene expression either by directly increasing the affinity of Runx1/AML1 to DNA, or by affecting the transcription initiation complex in a manner dependent on Runx1/AML1 binding to the AChE promoter, or even by an indirect mechanism involving additional, yet unknown components.

A second aspect of the present invention relates to a screening method/procedure for a candidate test drug, which is a modulator of the expression of any one of AChE variants and isoforms, which drug is then to be evaluated for its potential effect on the nervous system. In this screening method, a wide variety of substances will be screened, and those selected will be the test drugs applied in the method of the first aspect of the present invention.

Examples of substances that may be used in this screening include, but are not limited to peptides and proteins, hormones, growth factors, anticancer agents, antibacterial substances, anti-infective agents, anti-inflammatory drugs, antiviral agents, anesthetics, antifungal agents, antiparasitic agents, analgesics, smoking crave suppressants, drugs for male and female sexual disturbances, DNA and RNA and metal-based substances such as lithium and zinc.

The screening method of this second aspect of the invention comprises the steps of: (a) contacting a cell, preferably a neuron, with a test substance under suitable conditions; and (b) determining the effect of said substance on an end-point indication. This effect is defined as the capacity of a drug to modulate the expression and distribution of at least one of the AChE variants or isoforms. More specifically, the endpoint indication is the translocation of an ACHE variant or isoform within the cell, preferably in a neuron, from the cell body towards its processes.

Translocation into neuronal processes presumably depends on diverse cis-acting elements and/or secondary structures, primarily within the 3′ untranslated region [Wallace, C. S. et al. (1998) J Neurosci 18, 26-35; Blichenberg, A. et al. (1999) J Neurosci 19, 8818-29]. AChE-R mRNA includes no known dendritic targeting motifs; its targeting into dendrites may thus be associated with its unique 3′ sequence, or with the stress-induced accumulation of many more nascent ACHE-R mRNA transcripts. This is compatible with the intense nuclear labeling of AChE-R mRNA observed in Example 2 of this invention, reflecting a higher hnRNA:mRNA ratio for this transcript. The production of AChE-R might be controlled by the splicing regulator Nova through its intronic consensus motif, UCAUY. The brain-enriched polypyrimidine tract-binding protein (brPTB) may antagonize this Nova-generated alternative splicing [Polydorides, A. D. et al. (2000) Proc Natl Acad Sci USA 97, 6350-5], as both UCAUY and a possible polypyrimidine tract appear in conserved 3′ intronic locations of human (positions 7242-6 and 7220-45 respectively, GenBank Accession No. AF002993) and mouse (positions 13,242-6 and 13,210-44, AF312033) ACHE genes. The short half-life of AChE-R mRNA (ca. 4 hrs) [Chan, R. Y. et al. (1998) J Biol Chem 273, 9727-9733] also appears to be modified following stress. AChE-R mRNA includes a long U-rich element in the 3′ UTR positions 13,020-13,007 in GenBank Accession No. AF312033), that may contribute to mRNA destabilization through the binding of trans-acting proteins [J. Guhaniyogi and G. Brewer (2001) Gene 265, 11-23]. AChE mRNA has been shown to be stabilized during neuronal differentiation [J. Guhaniyogi and G. Brewer (2001) Gene 265, 11-23], the findings described herein suggest that this may be due, at least partially, to AChE-R transcripts being sequestered in neurites.

The preferred end-point indication for the AChE-R intracellular translocation is from the cell body to its processes, and vice-versa. Other acceptable translocations are to and from organelles to the cytoplasm and vice-versa, to and from the cytoplasm to the nucleus and vice versa, to and from the perikaryon to the cell body. Another possible indication is from within the cell to its periphery, since the monomeric ACHE-R isoform can be secreted.

The presence of AChE-R in neurites after long-term exposure to stimuli (Example 2) relates to the well-known phenomenon of sensitization following stress. Our electrophysiological recordings in the hippocampal CA1 region demonstrated weeks-long hypersensitivity of pre-stressed mice to anti-ChEs, attributing a role to the hippocampus in the cholinergic impact over post-stress sensitization. The increased sensitivity of the evoked response to atropine, a non-selective muscarinic antagonist, may likewise reflect a hyperexcitation-induced feedback response, which tightens hippocampal control over repeated stressful stimuli. The recorded synaptic field potentials were sensitive to both cholinergic and glutamatergic antagonists, suggesting a long-term change in the interactions between these two transmitter systems. Cholinergic-glutamatergic interactions have been associated with brain functions such as long-term potentiation, memory, and behavior [T. G. Aigner (1995) Curr Opin Neurobiol 5, 155-60], all of which are modulated following stress. The hypersensitized response may also be relevant to the additional functions attributed to neuritic AChE-R, especially non-catalytic capacities to compete with and mediate cell-cell and cell-matrix interactions. Neuroligin 1, for example, is a post-synaptic cell adhesion molecule of excitatory synapses [Song, J. Y. et al. (1999) Proc Natl Acad Sci USA 96, 1100-5], which includes an extracellular catalytically inactive AChE-homologous domain. When overexpressed, ACHE-R may possibly compete with neuroligin 1 for interaction with its partner protein neurexin □2 [H. Soreq and S. Seidman (2001) id ibid.], suggesting that excess ACHE-R may modify glutamatergic functions by impairing neurexin-neuroligin interactions.

In one embodiment, the ACHE variant expression and distribution is determined by in situ hybridization, utilizing ACHE mRNA variant specific probes. The probes to be used are nucleic-acid based probes. The visualization of the labeling reaction is possible by enzymatic, calorimetric, fluorescence or chemiluminescence means.

In another embodiment, the cells to be used in the screening are selected from the group consisting of PC12 cells, primary cerebellar neurons and any AChE-expressing primary neuron or established cell line, as for example glioblastoma and neuroblastoma cell lines.

In addition to its primary function of acetylcholine hydrolysis, AChE was shown to initiate adhesive cell-cell interactions through its core domain and promote mammalian neurite extension in a manner similar to that of its non-enzyme membrane protein homolog neuroligin [reviewed by Soreq and Seidman, (2001) Nat Rev Neurosci, 2, 294-302]. Nevertheless, the neuritogenic activities of distinct AChE variants appeared to depend on their unique C-termini, which to date were not found to share sequence homologies with other proteins. In hematopoietic cells, the 26 C-terminal residues of the stress-induced AChE-R protein exert proliferative and growth factor activities [Grisaru et al., (2001) Molecular Medicine, 7, 93-105]; however, it is not yet known whether these activities depend on extra- or intracellular interactions. Also, neither the molecular mechanism(s) nor the nervous system relevance of such activities were yet explored. The present invention reveals that at least part of the C-terminus specific non-catalytic effects of AChE-R are intracellular and PKCβII mediated.

As described in Example 3, co-immunoprecipitable and naturally co-localized complexes of AChE-R appeared to connect the C-terminal domain ARP1 with PKCβII and its intracellular shuttling protein RACK1. Intensified labeling and neuronal mobilization of both RACK1 and PKCβII was observed in the stress-protected transgenic nice constitutively overexpressing AChE-R. Transgenic ACHE-R overexpression translocated RACK1 to the perikaryal circumference and intensified PKCβII clustering in pyramidal neurons, and activated PKC suggesting functional relevance for AChE-R overexpression in the stress-suppressing cascades of neuronal signal transduction.

As shown by Example 3, AChE-R interaction with PKCβII is indirect and is mediated by the PKCβII shuttling protein RACK1. RACK1 is a member of the WD family of proteins. WD proteins can simultaneously bind different partners to various regions in their multi-blade rings [Smith et al. (1999) Trends Biochem Sci, 24, 181-5], which provides flexibility and combinatorial diversity. Thus, RACK1 is a scaffold for cell-cell interaction proteins such as □-integrin [Liliental and Chang, (1998) J Biol Chem, 273, 2379-83], members of signaling cascades like cAMP phosphodiesterase [Yarwood et al., (1999) J Biol Chem, 274, 14909-17], C2-containing proteins such as phospholipase C-□1 [Disatnik et al., (1994) Proc Natl Acad Sci USA, 91, 559-63], src kinase [Chang et al., (1998) Mol Cell Biol, 18, 3245-56] and PH domain-containing proteins such as the □-adrenergic receptor [Rodriguez et al., (1999) Biochemistry, 38, 13787-94]. Interaction between RACK1 and AChE-R would likely compete with other associations, changing the subcellular balance between these variable complexes. Alternatively, or in addition, AChE-R/RACK1 interactions may promote the formation of additional triple complexes with other proteins. Diverse links between AChE and other signaling molecules may, in turn, explain its capacity to exert various non-catalytic intracellular functions, a possibility which awaits further investigation.

PKCβII and RACK1 are readily available in neurons, albeit in inactive and possibly, non-associated or, loosely attached compositions. The present inventors have now found that stress responses and the subsequent accumulation of ACHE-R [Kaufer et al. (1998) id ibid.] facilitate the formation of triple, tightly bound AChE-R/RACK1/PKCβII complexes.

Thus a further aspect relates to the method of the invention for evaluating the effect of a test drug on the nervous system. According to this aspect, the test drug is selected by a screening method for a candidate substance which is a modulator of the interaction between AChE-R/RACK1/PKC. Of particular interest is a drug which is specifically airned at affecting CNS properties and treating CNS-associated disorders. This screening method comprises the steps of: (a) providing a reaction mixture comprising the AChE-R variant of acetylcholinesterase or any functional fragment thereof, the cognate receptor for activated kinase C (RACK1) and the protein kinase C □II (PKCβII); (b) contacting said mixture with a test drug under suitable conditions for said interaction; and (c) determining the effect of the test drug on an end-point indication. The effect of the test drug on the end-point is indicative of modulation of said interaction by the test drug.

In general, the present invention is directed to screening methods for identifying modulators of particular signal pathways. The interaction of the three complex participants (AChE-R/RACK1/PKC) is observed in the presence and absence of a candidate modulator. More particularly, the screening assay according to the invention is conducted by assessing the interaction between AChE-R, PKC and RACK1 either by measuring binding directly or by measuring a physiological or metabolic effect. The measurement is made in the presence and in the absence of a candidate modulator. Successful candidates which agonize the signal, effect an increase in a metabolic or physiologic output, whereas antagonists effect a decrease in a selected metabolic or physiological end point.

Depending on the assay system chosen, the interaction and its modification can be observed in a variety of ways, including intracellular binding assays affecting an observable parameter; either a physiological readout, such as change in subcellular distribution, or an in-vitro co-precipitation.

In one specific embodiment, the reaction mixture used by the method of the invention may be a cell mixture or a cell-free mixture.

According to a specific embodiment, this reaction mixture may optionally further comprise solutions, buffers and compounds which provide suitable conditions for interaction between AChE-R/RACK1/PKC and the detection of an end-point indication for said interaction.

In yet another specific embodiment the screened modulator may either inhibit or enhance the interaction between AChE-R/RACK1/PKC. Therefore, modification of the end-point indicates modulation of the interaction between AChE-R/RACK1/PKC by the test drug. For example, decrease or absence of the end point, in the presence of the test drug, indicates inhibition of the interaction between AChE-R/RACK1/PKC. Alternatively, presence or any increase of the end-point indicates enhancement of the interaction between AChE-R/RACK1/PKC by the test drug.

In a particular embodiment, the reaction mixture used by this screening method of the present invention is a cell-free mixture. According to this embodiment, the screening method comprises the steps of: (a) providing a cell free mixture comprising the AChE-R variant of acetylcholinesterase or any functional fragment thereof, RACK1 and PKCβII; (b) contacting said nixture with a test drug under conditions suitable for an in vitro interaction; and (c) determining the effect of the test substance on co-precipitation of PKCβII and RACK1 with the ACHE-R or fragment thereof as an end-point indication. Absence of said co-precipitation indicates inhibition of formation of a complex between AChE-R/RACK1/PKC by the test drug, whereas increase in said co-precipitation indicates enhancement of this complex formation.

In a particular embodiment, the cell-free mixture used by the method of the present invention comprises any one of AChE-R variant of acetylcholinesterase or any functional fragment thereof, RACK1 and PKCβII. These proteins may be provided as purified recombinant proteins or alternatively, as a cell lysate of cells expressing these proteins.

According to a specifically preferred embodiment, the ACHE-R variant of acetylcholinesterase comprised within the cell free mixture may be a fusion protein. A “fusion protein” as used herein is a recombinant protein made of segments, which are naturally not normally fused in the same manner. As a non limiting example such fusion protein may comprise AChE-R or functional fragment thereof and any one of GST (Glutathion-S-Transferase) and GFP (Qreen Fluorescent Protein, as exemplified in Example 3). Fusion protein of any of the three complex precipitant proteins may alternatively comprise protein such as MBP (maltose-binding protein). Thus, a fusion product of the AChE-R molecule with any one of GFP and GST, is a continuous protein molecule having sequences fused by a typical peptide bond, typically made as a single translation product and exhibiting properties derived from each source peptide.

As shown by Example 3, AChE-R/RACK1/PKCβII complexes are mobilized from their resting state intracellular location, translocating RACK1 to the perikaryal circumference and PKCβII into densely packed clusters. Modified properties and location of PKCβII in stress-responding neurons may possibly change the stress-induced kinase activation, phosphodiesterase mobilization, or other processes.

The presence of PKCβII in the deep cortical layers and in the stratum oriens and stratum radiatum of CA1 in hippocampus (FIG. 15) may reflect signal transmission across synapses between axons coming from outside the region. Staining in axon bundles such as the nigro-striatal tract may indicate that PKCβII is distributed along the entire axon of some neurons. Finally, the currently observed perikaryal punctiform pattern (FIG. 16), is compatible both with RACK1 interactions and with different functions, as considered below.

Transgenic ACHE-R-filled neurons with both punctiform PKCβII and RACK1 labeling are mostly relevant to stress-response inhibitory pathways [Herman and Cullinan, (1997) Trends Neurosci, 20, 78-84]. These were located in cortical upper layers, the hippocampal CA1 region, the lateral septum and the basolateral amygdala. PKCβII punctiform patterns also appeared in a subset of basolateral amygdala neurons, which are generally considered excitatory to psychological stress. However, the existence in this region of stress-inhibitory neurons, has been discussed with regards to the regulation of fearful behavior [Davis et al., (1994) Trends Neurosci, 17, 208-14]. These neurons presumably suppress other basolateral amygdala neurons that are stress-excitatory, consistent with the limited stress-related neuropathology hallmarks in the brain of AChE-R transgenic mice [Sternfeld et al. (2000) Proc Natl Acad Sci USA, 97, 8647-52]. The neuronal location of AChE-R/RACK1/PKCβII complexes further suggests relevance to the PKC-activated down regulation of transient K⁺ channels in dendrites of hippocampal CA1 pyramidal neurons [Hoffman and Johnston, (1998) J Neurosci, 18, 3521-8]. Such downregulation may contribute to the reduced stress overload of AChE-R overexpressing mice.

In yet another alternative embodiment, the reaction mixture used by the present invention is a cell mixture. More particularly, the cell mixture may be a transfected cell culture. According to this embodiment, the screening method comprises the steps of: (a) providing transfected cell culture expressing the AChE-R variant of acetylcholinesterase or functional fragment thereof, the cognate receptor for activated kinase C (RACK1) and the PKCβII; (b) contacting said transfected cell culture with a test substance; (c) detecting the interaction between AChE-R/RACK1/PKC in the presence of the test drug by searching for an end-point indication, whereby modification of said end-point indicates modulation of complex formation between AChE-R/RACK1/PKC by the test drug.

According to a particular embodiment, the transfected cell may be transfected by: (a) an expression vector comprising a nucleotide sequence coding for the ACHE-R variant of AChE or functional fragment thereof; and/or optionally (b), constructs comprising a nucleic acid sequence coding for any one of the cognate receptor for activated kinase C (RACK1) and the PKCβII.

“Expression Vectors”, as used herein, encompass vectors such as plasmids, viruses, bacteriophage, integratable DNA fragments, and other vehicles, which enable the integration of DNA fragments into the genome of the host. Expression vectors are typically self-replicating DNA or RNA constructs containing the desired gene or its fragments, and operably linked genetic control elements that are recognized in a suitable host cell and effect expression of the desired genes. These control elements are capable of effecting expression within a suitable host. Generally, the genetic control elements can include a prokaryotic promoter system or a eukaryotic promoter expression control system. Such system typically includes a transcriptional promoter, an optional operator to control the onset of transcription, transcription enhancers to elevate the level of RNA expression, a sequence that encodes a suitable ribosome binding site, RNA splice junctions, sequences that terminate transcription and translation and so forth. Expression vectors usually contain an origin of replication that allows the vector to replicate independently of the host cell.

The term “operably linked” is used herein for indicating that a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

Plasmids are the most commonly used form of vector but other forms of vectors which serve an equivalent function and which are, or become, known in the art are suitable for use herein. See, e.g., Pouwels et al. (1985 and supplements) Cloning Vectors: a Laboratory Manual, Elsevier, N.Y.; and Rodriquez, et al. (eds.) (1988) Vectors: a Survey of Molecular Cloning Vectors and their Uses, Buttersworth, Boston, which are incorporated herein by reference.

Accordingly, the term control and regulatory elements includes promoters, terminators and other expression control elements. Such regulatory elements are described in Goeddel; [Goeddel et al. (1990) Gene Expression Technology: Methods in En zymology 185, Academic Press, San Diego, Calif.]. For instance, any of a wide variety of expression control sequences that control the expression of a DNA sequence when operatively linked to it may be used in these vectors to express DNA sequences encoding any of the three desired proteins used by the screening method of this invention.

In general, such vectors contain in addition specific genes, which are capable of providing phenotypic selection in transformed cells. A variety of selectable markers can be incorporated into any construct. For example, a selectable marker which confers a selectable phenotype such as drug resistance, nutritional auxotrophy, resistance to a cytotoxic agent or expression of a surface protein, can be used. The expression vector of the invention may further comprise a tag sequence. Such sequences enable the detection and isolation of the recombinant protein. As a non-limiting example such tag sequences may be any one of HA, c-myc, GST, GFP and His-6.

The use of prokaryotic and eukaryotic viral expression vectors to express the genes coding for the ACHE-R and optionally for the PKCβII and the RACK1 according to the present invention, is also contemplated.

The vector is introduced into a host cell by methods known to those of skilled in the art. Introduction of the vector into the host cell can be accomplished by any method that introduces the construct into the cell, including, for example, calcium phosphate precipitation, microinjection, electroporation or transformation. See, e.g., Ausubel, F. M. ed. (1989) Current Protocols in Molecular Biology, John Wiley & Sons, New York.

“Cells” or “transfected cells” are terms used in the present invention. It is understood that such terms refer not only to the particular subject cells but to the progeny or potential progeny of such a cell. Because certain modification may occur in succeeding generations due to either mutation or environmental influences, such progeny may not, in fact, be identical to the parent cell, but are still included within the scope of the term as used herein.

“Transfected cell” as used herein refers to cells which can be stably or transiently transfected with vectors constructed using recombinant DNA techniques. A drug resistance or other selectable marker is intended in part to facilitate the selection of the transformants. Additionally, the presence of a selectable marker, such as drug resistance marker may be of use in keeping contaminating microorganisms from multiplying in the culture medium. Such a pure culture of the transfected cell would be obtained by culturing the cells under conditions which require the induced phenotype for survival.

As used herein, the term “transfection” means the introduction of a nucleic acid, e.g., an expression vector, into a recipient cells by nucleic acid-mediated gene transfer.

It is to be appreciated that the cell or the transfected cell used by the screening method of the present invention may further contain an endogenously or exogenously expressible possible interactor molecules essential for activation of said pathway or said complex formation.

One preferred end-point indication for the “cell mixture” and preferably the transfected cell based screening method, may be the subcellular translocation of catalytically active PKCβII. Subcellular distribution of PKCβII can be detected by a visually detectable signal, generated for example, by using in situ hybridization or immunohystochemistry. A visually detectable signal may be for example a fluorescent signal (FIG. 16 using confocal microscopy).

In general, PKC is mainly referred to as a morphologically active kinase. However, PKCβII has been shown to be associated with oxidative [Paola et al., (2000) Biochem Biophys Res Commun, 268, 642-6] and ischemic stresses [Cardell and Wieloch, (1993) J Neurochem, 61, 1308-14] and essential for fear conditioning [Weeber et al., (2000) J Neurosci, 20, 5906-14]. Intriguingly, genomic disruption of the glucocorticoid receptor, which upregulates ACHE-R production [Grisaru et al., (2001) ibid] abolished anxiety responses [Tronche et al., (1999) Nat Genet, 23, 99-103]. The current findings of the present invention therefore propose a chain of events that may assist in overcoming traumatic stress responses. This cascade initiates with glucocorticoid hormone release, proceeds with transcriptional activation and alternative splicing to elevate AChE-R levels and results in RACK1 and PKCβII mobilization. AChE-R thus emerges as a modulator and PKCβII—an initiator of long-term morphological responses to stress. Relevant processes include the reported PKC-induced inhibition of presynaptic metabotropic glutamate receptors [Macek et al., (1998) J Neurosci, 18, 6138-46], coupling to the cAMP response element binding protein in CA1 neurons [Roberson et al., (1999) J Neurosci, 19, 4337-48] and the PKC-regulated release of vesicle pools [Stevens and Sullivan, (1998) Neuron, 21, 885-93]. The increase in neuronal PKCβII in AChE-R transgenic mice further proposes that when such changes become permanent they can confer stress protection, whereas the PKCβII disruption study indicates that chronic impairments in this cascade may be detrimental. Nevertheless, excessive AChE-R production may also be detrimental, at least for recovery from closed head injury [Shohami et al., (2000) J Mol Med, 78, 228-36].

Similarly to the “cell-fiee mixture”-based screening method, another preferred end-point indication for the “cell mixture” based screening method, may be co-precipitation of PKCβII and RACK1 with the ACHE-R or functional fragment thereof. Co-precipitation of these interacting molecules leads to a detectable signal, whereby modification of said detectable signal in the presence of the test drug indicates modulation of the formation of a complex between AChE-R/RACK1/PKC by said test drug.

In yet another specifically preferred embodiment, the cell or the transfected cell used by the screening method of the invention may be a prokaryotic or eukaryotic cell, particularly a bacterial cell, yeast cell, an insect cell, a plant cell or preferably a mammalian cell. Most preferred are cells selected from the group consisting of COS and PC12 cells.

The test drug for the screening and evaluation methods of the invention may be any substance selected from the group consisting of protein based, carbohydrates based, lipid based, nucleic acid based, natural organic based, synthetically derived organic based, and antibody based substances.

As used herein, the term “nucleic acid” refers to polynucleotides such as deoxyribonucleic acid (DNA), and, where appropriate, ribonucleic acid (RNA). The terms should also be understood to include, as equivalents, analogs of either RNA or DNA made from nucleotide analogs, and, as applicable to the embodiment being described, double-stranded polynucleotides and single-stranded such as sense or antisense. More particularly, anti-sense directed against the AChE-R variant may attenuate said complex formation.

Among successful candidate drugs or substances will be peptides which mimic regions on either of the three complex forming proteins, as well as non-peptidic small molecules. Due to their ease of identification, these peptides are particularly useful in alternate forms of the screening assays that detect modification and modulation of the interaction between AChE-R/RACK1/PKC. Although the assay methods disclosed may not all be suitable for direct screening of large chemical libraries, they do enable a sophisticated screening of candidates that can be combined with other techniques for selecting leads.

In a particular embodiment a protein or antibody based substance may be a product of a combinatorial library. Thus, the invention is also directed to methods to screen libraries of candidate modulators using the above-described methods and to peptides representative of sites on any of the three complexed proteins, which are themselves useful in these assays as well as in other applications involving the relevant interaction.

Based on the above results, AChE-R and/or PKCβII levels should be also tested in patients with post-traumatic stress disorder [McEwen, (1999) Annu Rev Neurosci, 22, 105-22], post-stroke phenomena and inherited susceptibility to processes in which PKCβII plays a major role, e.g. panic attacks [Gorman et al., (2000) Am J Psychiatry, 157, 493-505], where fear conditioning is intimately involved. Likewise, the dissociation between RACK1 and PKCβII under ethanol exposure [Ron et al. (2000) J Biol Chem, 274, 27039-46] may be relevant to the stress-suppressing effect of alcohol. In addition, this study calls for testing PKCβII levels and subcellular localization in patients hypersensitive to anticholinesterases (e.g Alzheimer's disease drugs) which also induce AChE-R overproduction [Kaufer et al., (1998) ibid; Shapira et al. (2000) Hum Mol Genet, 9, 1273-81].

Therefore, candidate drug which modulates the interaction between AChE-R/RACK1/PKCβII, may affect fear conditions, panic and traumatic stress responses.

Triple AChE-R/RACK1/PKCβII complexes are likely involved with the neuronal redistribution of PKCβII n brain development [Gallicano et al., (1997) Bioassays, 19, 29-36], aging [Battaini et al., (1999) Exp Neurol, 159, 559-64] and neurodegeneration [McNamara et al., (1999) J Neurochem, 72, 1735-43], all of which involve considerable modulations in AChE-R levels. AChE-R is further expressed in other RACK1/PKCβII producing tissues, including epithelial, muscle, hematopoietic, and germ cells [Soreq and Seidman, (2001) id ibid] where its capacity to induce PKCβII—mediated changes should be examined.

Disclosed and described, it is to be understood that this invention is not limited to the particular examples, process steps, and materials disclosed herein as such process steps and materials may vary somewhat. It is also to be understood that the terminology used herein is used for the purpose of describing particular embodiments only and not intended to be limiting since the scope of the present invention will be limited only by the appended claims and equivalents thereof.

It must be noted that, as used in this specification and the appended claims, the singular forms “a”, “an” and “the” include plural referents unless the content clearly dictates otherwise.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

Experimental Procedures

Plasmid constructs: All plasmids were under CMV control.

AC6—AChE expression construct, containing the proximal promoter, intron I and exons E2-E6 [Shapira (2000) id ibid.].

WtAC6 and □AC6—AC6 plus 200 bp from its distal enhancer (wild type and mutated sequences, respectively) [Shapira (2000) id ibid.].

Runx1/AML1 and t-Runx1/AML1—construct containing the sequence encoding the intact murine protein and truncated Runx1/AML1, respectively, without the trans-activating domain encoding sequence [Ben Aziz Aloya, R. B. et al. (1998) Cell Death Differ. 5, 765-73].

p300—construct containing the sequence encoding the co-activator p300 (Accession Number U01877), as previously described [Eckner, R. et al. Genes Dev 8:869-84 (1994)] (kindly provided by Dr. R. H. Goodman, Portland, Oreg.).

GFP-Fused AChE Variant Expression Constructs:

pGARP—fusion protein of GFP and the C-terminal peptide of hAChE-R, including a fragment of the human ACHE-R cDNA (nt 1796-18G5 of hAChE, Accession Number M55040), followed by nt 1-111 from the genonmic hAChE I4-E5 domain (Accession S71129, stop codon at position 86) cloned into the Bsp120I/XbaI sites of pEGFP-C2 (Clontech). The following peptide is encoded:

PLEVRRGLRAQACAFWNRFLPILLSATGMQGPAGSGWEEGSGSPPGVTPLFSP (SEQ ID NO:1).

PGASP—fusion protein of GFP and the C-terminal peptide of the synaptic form of human AChE, including a fragment of AChE cDNA (nt 1794-2001, Accession M55040) inserted into the EcoR I/Sal I sites of pEGFP-C2 (Clontech). The following peptide is encoded:

RPLEVRRGLRAQACAFWNRFLPKLLSATDTLDEAERQWKAEFHRWSSYMVHWKNQFDHYSKQDRCSDL (SEQ ID NO:2).

DGASP—fusion protein of GFP and a mutated C-terminal peptide of the synaptic form of human AChE, created by inserting a fragment of AChE cDNA (nt 1794-2001, Accession M55040) containing a frame-shift mutation into the EcoR I/Sal I sites of pEGFP-C2 (Clontech). The frame-shift mutation results in a change of the final 8 amino acids of the C-terminal peptide sequence into RLAAQTCD (SEQ ID NO:3).

Antibodies: Immunohistochemical analysis were essentially as previously described [Shoham, S. and Ebstein, R. P. (1997) Exp Neurol, 147, 361-76; Sternfeld et al. (2000) id ibid.], using rabbit anti-ARP 1:100, rabbit anti PKCβII (Cat. No. sc-210, Santa Cruz) 1:100, rabbit anti PKCβII (Sigma-Rehovot, Israel) 1:250 and mouse anti RACK1 (Cat. No. R20620, Transduction Laboratories, San Diego, Calif.) 1:200. Immunoblot analysis were with rabbit anti-N-terminus ACHE antibodies (Cat. No. N-19, Santa Cruz), 1:500; mouse monoclonal antibody against all isoforms of PKC of mouse, rat and human origin (Cat. No. sc-80, Santa Cruz), dilution 1:100, or mouse monoclonal antibody against RACKI (R20620, Transduction Laboratories), dilution 1:2500. Polyclonal goat anti-human AChE “synaptic” C-terminal peptide (Santa Cruz Biotechnology, Inc.) was used at 1:100 [Sternfeld (2000) id ibid.] was used at 1:100, polyclonal rabbit anti-human Runx1/AML1 was used at 1:250 (for immunohistochemistry and immunocytochemistry), biotinylated donkey anti-goat and donkey anti-rabbit (Chemicon, Temecula, Calif., USA) were used at 1:200, horseradish peroxidase conjugated goat anti-rabbit and donkey anti-goat (Jackson Immunoresearch Laboratories, West Grove, Pa., USA) were used at 1:200 and 1:10,000 for immunohistochemistry and overlay assays, respectively.

General Methods in Molecular Biology

A number of methods of the molecular biology art are not detailed herein, as they are well known to the person of skill in the art. Such methods include PCR cloning, expression of cDNAs, analysis of recombinant proteins or peptides, transformation of bacterial and yeast cells, transfection of mammalian cells, and the like. Textbooks describing such methods are, e.g., Sambrook et al., Molecular Cloning, A Laboratory Manual, Cold Spring Harbor Laboratory, ISBN: 0879693096, 1989; Current Protocols in Molecular Biology, by F. M. Ausubel, ISBN: 047150338X, John Wiley & Sons, Inc. 1988; and Short Protocols in Molecular Biology, by F. M. Ausubel et al. (eds.), 3rd ed., John Wiley & Sons, ISBN: 0471137812, 1995. These publications are incorporated herein in their entirety by reference. Furthermore, a number of immunological techniques are not in each instance described herein in detail, as they are well known to the person of skill in the art. See, e.g., Current Protocols in Immunology, Coligan et al. (eds), John Wiley & Sons. Inc., New York, N.Y.

Sequence data analysis: Transcription factor binding sites were sought in a cosmid clone (GenBank accession no. AF002993) spanning the human (h)ACHE gene and 22 kb of its upstream sequence, using the MatInspector 2.0 program [Quandt, K. et al. Nucleic Acids Res 23:4878-84 (1995)] (core similarity of 1, matrix similarity of 0.85) and Find-Patterns, of the University of Wisconsin software package.

Recombinant RACK1 preparation: A plasmid overexpressing MBP-RACK1 in E. coli pDEM31, a derivative of pMAL-c2 (New England Biolabs, Beverly, Mass.) [Rodriguez, M. M. et al. (1999) Biochemistry 38, 13787-94], was a kind gift from Dr. Daria Mochly-Rosen, Stanford. The pDEM31 vector was introduced into E. coli, and recombinant RACK1 fused to the maltose binding protein was purified on an amylose affinity column (New England Biolabs). The 36 kDa RACK1 protein was released by proteolysis with Factor Xa (New England Biolabs).

Coimmunoprecipitation. Clear supernatants of PC12 or COS cell homogenates (200 □L, 1.5 mg protein/mL) were prepared by manual homogenization, followed by 30 min centrifugation at 12,000×g, 4° C. Supernatants were diluted five fold with NET buffer (50 mM Tris-HCl, pH 7.4, 150 mM NaCl, 1 mM EDTA, 0.25% gelatin and complete mini protease inhibitors cocktail (Roche)), in a final concentration of 0.05% Triton X-100. Goat polyclonal antibodies (Santa Cruz) targeted to the N-terminal domain. of hAChE (10 □L, 200 □g/mL) were added for overnight rotation at 4° C. 75 □L of Protein G MicroBeads (Miltenyi Biotec, Bergisch Galdbach, Germany) was added and incubation continued for another hour. Mixtures were loaded on MACS magnetic separation columns (Miltenyi Biotec), washed 3 times with 200 □L of TBS buffer containing 0.05% Tween-20 and eluted with gel loading buffer. Elutes were separated by sodium dodecyl sulfate polyacrylamide gel electrophoresis (SDS-PAGE) (Bio-Rad, Hercules, Calif.), blotted and incubated with the noted detection antibodies. For immunoprecipitation, dissected mouse brain regions were homogenized in 9 volumes of lysis buffer. Homogenates were passed several times through a 21G needle. Insoluble debris was removed by a 30 min 12,000×g centrifugation. Homogenates were kept frozen at −70° C. until use.

Western Blot: Western Blots were carried out in the classical method described in ‘Molecular Cloning—A laboratory manual by Sambrock et al., Cold Spring Harbor Laboratory Press, 2^{nd} edition, 1989’.

In situ hybridization: High resolution in situ hybridization in 50 □m paraffin sections was performed using 5′ biotinylated, 2-O-methylated cRNA probes selective for the “synaptic” (AChE-S mRNA) and “readthrough” (AChE-R mRNA) transcripts of AChE [Grisaru (1999) id ibid.]. In addition, we used the following probes:

(SEQ ID NO:4)

5′CGAGUUGCUAUGGCUGCCCUCGGUCUCCACCACGUCGCUCUGGCU
GGGGA-3′;

(SEQ ID NO:5)

5′GGGUGAAAUGGGTGUCGCTGGGTGGACAGAGGAAGAGGTGATGGAT
CCCA-3′
[Runx1/AML1 mRNA (Accession Number NM009821)];

(SEQ ID NO:6)

5′CUGACACGGUCUUCACCAUUCCCGCUCUGGCGUAAGCCCCAGCAGA
CTGG-3′
[c-fos mRNA (Accession Number V00727)]; and

(SEQ ID NO:7)

5′UUCAUCCCCUGGCUGGCGUUCAUGUUGCUCACGGAAGAGUAGCCC
UCAGG-3′
[rHNF3b mRNA (Accession Number NM012743.1)].

Staining was revealed using alkaline phosphatase/streptavidin conjugate and Fast Red (Boehringer) as a substrate. Nuclei were counterstained with hematoxylin (Sigma).

Double labeling fluorescent in situ hybridization (FISH) for analysis of sub-cellular structures. Poly-L-ornithine coated coverslips (0.5 mg/ml, 10 min, RT) were sterilized by UV irradiation (TUV/c 8 Watts, 3 hrs at RT). Coverslip-grown cells were fixed in 3% paraformaldehyde (20 min), dried (37° C., 1 hr), washed 2×5 min in phosphate-buffered saline (PBS) and 2×5 min in 0.2% Tween-20 PBS (PBT), incubated with 10 mg/ml proteinase K (5 min, RT), and re-washed (PBT, 2×5 min). Streptavidin (1 mg/ml, 30 min, room temp) served to block non-specific labeling. Hybridization was in a humidified chamber with 10 □g/ml probe in 50% formamide, 5×SSC, 10 mg/ml tRNA, 10 mg/ml heparin (90 min, 52° C.). Washes were in 50% formamide, 5×SSC and 0.5% sodium-dodecyl-sulfate (SDS), and in 50% formamide, 2×SSC at 60° C., then at room temp, in Tris-buffered saline with 0.1% Tween-20 (TBST) including 2 mM levamisole (Sigma). Following blockade in 1% skim milk (30 min), digoxigenin-labeled probes were detected with fluorescein- or rhodamine-labeled anti-DIG antibodies (1 hr, room temp, 3 washes in TBST). Biotin-labeled probes were detected with streptavidin-Cy2, Cy3 or Cy5 conjugates (CyDye™, Amersham Pharmacia Biotech, Little Chalfont, UK), (30 min, room temp, similar washes). Signal production involved a streptavidin-alkaline-phosphatase conjugate (Amersham) diluted 1:50 in TBST (30 min, room temp, 3 washes in TBST with 2 mM levamisole) and incubation in Fast Red substrate (Roche Diagnostics, Mannheim, Germany) in 0.1 M Tris-HCl pH 7.4. Mounting was with IMMU-MOUNT (Shandon Inc, Pittsburgh, Pa., USA).

Fluorescence Double Labeling for Immunocytochemistry:

RACK1 and ARP: Primary staining solutions contained 0.001% trypsin inhibitor (Sigma type IIS), 0.3% Triton X100, 0.05% Tween 20, 2% normal goat serum, 2% normal donkey serum, rabbit anti-ARP1 (1:100) and mouse anti-RACK1 (1:100). The secondary antibody solution contained 0.3% Triton X100, 0.05% Tween 20, 2% normal goat serum, 2% normal donkey serum, donkey-anti-rabbit conjugated to fluorescein (Chemicon, AP182F) diluted 1:100 and goat-anti-mouse conjugated with tetra-methyl-rhodamine (Sigma, T7782) diluted 1:800. Sections were mounted on SuperFrost slides (Menzel Glaser, Freiburg, Germany), air-dried, covered in ImmuMount (Shandon, Pittsburgh, Pa.) and covered for microscopy.

PKCβII and ARP1: The primary staining solution contained 0.3% Triton X100, 0.05% Tween 20, 2% normal goat serum, 2% normal donkey serum, rabbit anti-ARP (1:100) and mouse-anti-PKβII (Sigma, P8083), diluted 1:500. Secondary antibody solutions and preparation for microscopy were as specified above for ARP and RACK1.

Quantification of the fluorescent in situ hybridization signal: In situ hybridization was performed as described above. The detection method using rhodamine labeled antibodies does not include an amplification step of the signal and thus offers the possibility of semi-quantitative analysis of the mRNA. The sections were scanned on the confocal microscope (Bio-Rad MRC-1024 confocal scanhead, Hemel Hemsted Herts, UK) and compound images were achieved by maximum value projection of 17 images. The intensity of the probe signal in each preparation was measured with the image analysis software Image Pro Plus (Media Cybernetics, USA).

Image analysis: Photography was carried out using a Zeiss Axioplan microscope, equipped with a digital camera. Red staining efficiency on the cytoplasmic regions of scanned images were evaluated using Adobe Photoshop 4.0 (Adobe Systems, Inc. San Jose, Calif.) at 240 output levels. Background values were subtracted and findings expressed as mean±standard error for 10-20 cells per sample (4-6 samples per grade). Student's t-test was applied for variance analysis.

Confocal microscopy: A Bio-Rad MRC-1024 confocal scanhead (Hemel Hempsted Herts., UK) coupled with an inverted Zeiss Axiovert 135 microscope equipped with a plan apochromat 40×/1.3 oil immersion objective was used for confocal imaging. GFP and Fast-Red were excited at 488 nm and emissions were collected through a 525±20 nm filter for GFP and a 580 nm±16 nm filter for Fast Red. The confocal iris was set at 3 mm. Sections were scanned every 0.5 mm. Images were merged using the Image Pro Plus software (version 4.0, Media Cybernetics, Silver Spring, Md.).

Confocal microscopy for detecting RACK1/PKC/AChE-R complexes: Slices were scanned using a Bio-Rad MRC-1024 scanhead (Hemel Hempsted Herts., UK) coupled to an inverted Zeiss Axiovert 135M microscope with a 40×oil immersion objective (N.A. 1.3). Excitation wavelength was 488 nm (using 10% of a 100 mW laser power). Fluorescence emission was measured using a 580df32 bandpass interference filter (580 nm±16 nm) for detecting tetra-methyl-rhodamine and a 525/40 filter for detecting fluorescein. The confocal iris was set to 3 mm. Conditions of scanning took into consideration the overlap of fluorescein fluorescence into the rhodamine filter (as were determined by control experiments). Images were then further processed using Image pro Plus 4.01 program (version 4.0, Media Cybernetics, Silver Spring, Md.).

Primary tumor sections: Formalin fixed and paraffin embedded surgical specimens were obtained with the authorisation of the Tel Aviv Sourasky Medical Center Committee for Human Experimentation Helsinki Committee). 5 □m sections of astrocytoma tumors were stained with hematoxilin-eosin and clinically graded according to the St. Anne-Mayo grading system (grades I-IV), based on nuclear atypia, mitoses, vascular proliferation and necrosis [Daumas-Duport (1988) id ibid.].

Immunocytochemistry: Formalin fixed and paraffin embedded 5 μm sections of astrocytoma tumors and metastatic brain melanoma were stained using rabbit antisera against either AChE-S, ACHE-R or the Runx1/AML1 protein, and secondary antibodies conjugated to horseradish peroxidase, as previously described [Sternfeld, M. et al. (2000) id ibid.]. Transfected COS-1 cells were fixed with 4% paraformaldehyde for 15 min at room temperature (RT), washed in PBS, incubated in 0.1M glycine buffer (in PBS, 10 min at RT), dried overnight, washed twice with PBS and further handled as described for tissue immunohistochemistry.

For RACK1 staining, sections were incubated with the primary antibody and then with biotin-conjugated donkey anti-rabbit antibody (Cat No. AP132B, Chemicon, Temecula, Calif.; 1 hour, room temp., overnight at 2-8° C.) and extravidin-peroxidase (Sigma). RACK1 staining was further preceded by trypsin type II treatment (Sigma), 1 □g/ml with calcium chloride 0.001% for 2 min, at room temp., which required the addition of 0.001% soybean trypsin inhibitor (Sigma) during staining. Detection was with horseradish peronidase-conjugated goat anti-mouse antibody (1:100 dilution, Sigma). Pre-incubation of anti-RACK1 with 10 □M RACK1 for 1 hour at room temperature totally eliminated staining with anti-RACK1, demonstrating specificity. For all antibodies, staining was intensified with 0.075% diaminobenzidine and 0.05% nickel ammonium sulfate

Cell culture and transfections: COS-1 cells were grown in humidified chambers in Dulbeco's modified Eagle's medium (Biological Industries, Beit Ha'emek, Israel) supplemented with 10% fetal calf serum (FCS) and 2 mM L-glutamine at 37° C., 5% CO₂. Transfections of the cells with 2 mg plasmid DNA per well were carried out using Lipofectamine Plus (Gibco BRL Life Technologies, Bethesda, Md.) according to the manufacturer's instructions. AChE activity assays, used as reporter for transcription efficiency, were performed in cell homogenates prepared 24 hours post-transfection in PBS containing 1% Triton X-100. In addition, COS-1 cells were co-transfected with the Runx1/AML1 construct together with either the GASP or the GARP expression constructs. Expression and intracellular localization of GASP, GARP and RUNX1/AML proteins were evaluated through immunocytochemistry and confocal microscopy.

PC12 cells were transiently transfected with the plasmid encoding AChE-R, using Lipofectamine Plus (Life Technologies, Paisley, UK). Cells were lysed 24 h following transfection in lysis buffer (0.1M phosphate buffer pH 7.4, 1% Triton X-100, and complete mini protease inhibitor cocktail) (Roche, Mannheim, Germany). Cell debris was removed by centrifugation at 12,000×g for 10 min.

Tissue Homogenizations: Tissues from various sources were homogenized in either LSD or solution-D buffer in an Eppendorf tube using a hand-held, battery-powered homogenizer. Homogenates were microfuged for 30 min at 4° C. and the clear supernatant transferred to a clean tube. Homogenates were assayed for enzymatic activity. In some cases, Enzyme Antigen Immunoassay (EIA) was employed to distinguish between rHAChE and background material in the fractions.

AChE Activity and Inhibition Assays:

a. Activity Assays: AChE activity was evaluated in all experiments using a modified version of the colorimetric assay devised by Ellman et al. (1961) adapted to a 96-well microtiter plate. 10-20 □l of enzyme was assayed in 200 Ξl (final volume) 0.2M phosphate buffer (pH 7.4), 0.5 mM 5-5′ dithiobis-nitrobenzoic acid (DTNB) containing 1 mM acetylthiocholine iodide (Sigma) substrate.

b. Inhibition Assays: Samples were pre-incubated with specific inhibitors 20-40 minutes prior to the addition of substrate. A₄₀₅ was monitored at 0.5 min intervals for 5-10 minutes using a VMAX microtiter plate reader (Molecular Devices Corporation (MDC), Palo Alto) and the rate of hydrolysis was calculated by regression analysis using a dedicated software package (SoftMax, MDC). All assays were performed at room temperature. Raw data was translated into activity units (nanomoles of hydrolyzed substrate per embryo per hour) using the extinction coefficient for DTNB=13,600 M⁻¹cm⁻¹ and a path length of 0.5 cm. The inhibition assays were performed in tissue homogenates or immunoadsorption-purified enzyme preparations, as described below. For reversible inhibitors, the concentration range included six orders of magnitude and the Kᵢ values were calculated by the method of Hobbiger and Peck [Bailey, C. H. et al. (1996) Proc Natl Acad Sci USA 93, 13445-52], based on a Kₘ value of 0.14 mM ATCh for human ACHE [0. Steward and E. M. Schuman (2001) Annu Rev Neurosci 24, 299-325; H. Soreq and S. Seidman (2001) id ibid.]. For irreversible inhibitors, we used an antibody-coated microtiter plate assay as described [H. Kang and E. M. Schuman (1996) Science 273, 1402-6].

Protein concentrations were determined using the Bio-Rad DC Protein Assay kit (Bio-Rad Laboratories, Hercules, Calif., USA).

Sucrose gradient ultracentrifugation: Sucrose gradient was performed in 20-80 μl of each sample, provided that the total AChE activity would be sufficient to achieve a ΔA₄₀₅ of approximately 2.4/min in the Ellman assay. This amount of activity enabled the analysis of the more dilute sedimentation fractions. Muscle extracts were loaded with high salt buffer, while somatosensory cortex extracts were loaded with low salt buffer. Samples were applied to 12 ml of 5-20% linear sucrose density gradients. The samples underwent ultracentrifugation for 20 hr at 240,000×g at 4° C., in the presence of bovine catalase (Sigma) as a sedimentation marker (11.4 S). Fractions were collected in 96-well microtiter plates and assayed for AChE activity essentially as described above.

Surgical implantation of a dyalisis probe: Implantation of a dyalisis probe in the experimental animal was performed according to the method described in Erb (2001) [Erb et al (2001) J Neurochem 77, 638-646].

Laboratory Animals and Stress Experiments: 6-8 weeks old FVB/N male mice were subjected to saline injection (0.2 ml, intraperitoneal) which induces mild psychological stress in this stress-sensitive strain. Stressed mice, control and ACHE-R transgenic mice [Sternfeld et al. (2000) id ibid.] were sacrificed 24 h post-injection. To prepare brain sections, four mice from each line were deeply anesthetized with Pental (pentobarbitone sodium 200 mg/ml, CTS Chemical industries, Petah Tikva, Israel) at a dose of 100 mg/Kg and transcardially perfused with 4% (vol/vol) paraformaldehyde. Brains were post-fixed by immersion in 4% (vol/vol) paraformaldehyde (overnight, 2-8° C.) and incubated in 12% (vol/vol) sucrose in 0.1M phosphate buffered saline (PBS). Coronal cryostat sections (30 μm) were floated in PBS and kept at −20° C. in 40% (vol/vol) ethylene glycol and 1% polyvinylpyrrolidone in 0.1 M potassium acetate (pH 6.5) until staining.

### Example 1

Muscarinic Receptor Mediates Alternative Splicing Shift of AChE mRNA

To determine whether cholinergic neurotransmission is involved in shifting the alternative splicing of ACHE mRNA under stress, and to compare the consequences of stress to those of anti-AChE exposure, the inventors developed a system combining molecular, cell biology and biochemistry approaches.

1A. AChE Activity

In the brain of naive rats, hydrolytic AChE activities were 62 and 82 nmol/min/mg protein for the parietal cortex and hippocampus, respectively. Within 3 and 24 hr, PG/S injection induced a minor, insignificant increase (>10%) in hydrolytic AChE activities in the hippocampus and the parietal cortex (Table 1). However, 3 hr following injection of the irreversible anti-AChE DFP, AChE activity was reduced to ca. 46 and 69% of control in the hippocampus and parietal cortex, respectively. By 24 hr post-treatment, hippocampal AChE levels were increased to 80% of naive animal levels whereas in the cortex, activity declined to 57% of control. Thus the post-exposure process of nascent AChE synthesis appeared to be brain region-dependent.

Table 1

Muscarinic Antagonists Modify the Recovery of

AChE activities following anti-AChE exposure

| Cortex | | | Hippocampus | |
|---|---|---|---|---|
| treat- | AChE activity, nmol/min/mg^{a} | | AChE activity, nmol/min/mg^{a} | |
| ment | % naïve | | % of naive | |
| 3 hr | | | | |
| DFP | 43 ± 4.8 | 69* | 39 ± 13.3 | 46 |
| scop+ | | | | |
| DFP | 36 ± 4.4 | 58* | 37 ± 15.9 | 44 |
| pir + D | | | | |
| FP | 21 ± 2.7 | 34*/** | 36 ± 12 | 42 |
| PG/S | 63 ± 25 | 101 | 88 ± 8.6 | 108 |
| PO | 65 ± 17 | 103 | 87 ± 3.8 | 106 |
| 24 hr | | | | |
| DFP | 35 ± 15 | 57 | 68 ± 15.7 | 80 |
| scop+ | | | | |
| DFP | 45 ± 10 | 72 | 71 ± 17.3 | 83 |
| pir + D | | | | |
| FP | 37 ± 5.2 | 59 | 37 ± 15.9 | 44* |
| PG/S | 68 ± 11 | 110 | 85 ± 2.6 | 100 |
| PO | 71 ± 14 | 114 | 96 ± 3.5 | 117* |
| naive | 62 ± 6.4 | 100 | 82 ± 3.1 | 100 |

^{a}AChE hydholytic activities measured in cortices and hippocampus of rats treated with the noted protocols (n = 3-4 in each group) at the noted times post-treatment.
*p <0.05 in comparison to the naive group
**p <0.05 in comparison to the DFP plus vehicle group
Abbreviations: scop., scopolamine; pir., pirenzepine; PO, octylpyridostigmine;

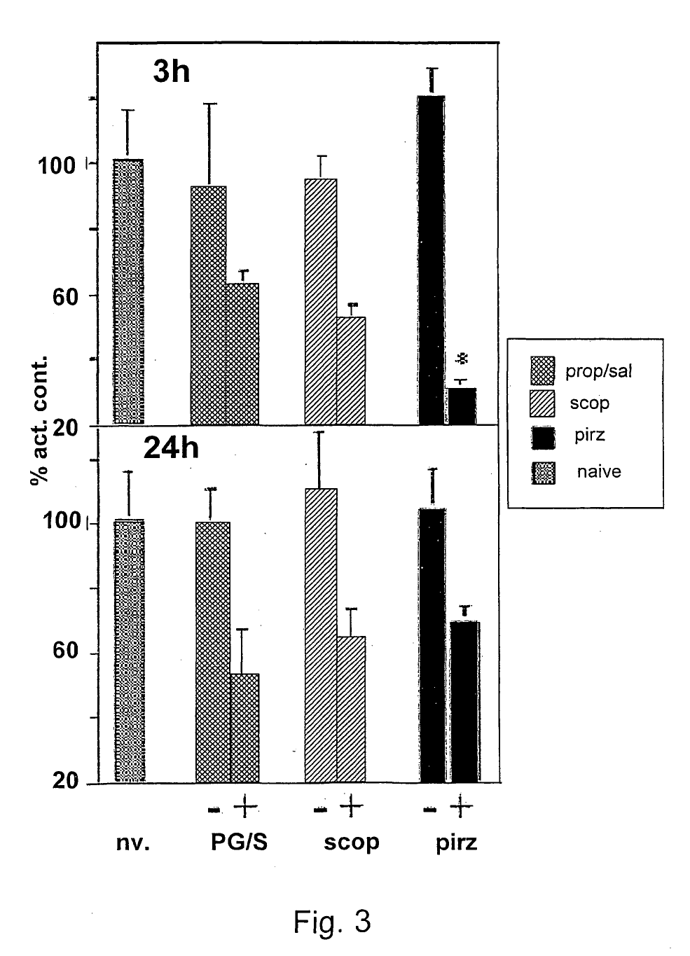

Pre-treatment with the M1 muscarinic antagonist pirenzepine exacerbated the cortical DFP-induced decrease in AChE activity down to 44% of control after 3 hours. Hippocampal inhibition remained unchanged at this time-point, but a significant decrease was noticed in the hippocampus after 24 hours when regaining of enzyme activity was limited to 63% of control. Exposure to octylpyridostigmine (PO) resulted in a 17% increase of total AChE activity after 24 hr, demonstrating drug-dependent differences in this modulation. In contrast, the non-selective muscarinic inhibitor scopolamine had no significant effect on the DFP-induced decrease in AChE activities either 3 or 24 hours post-treatment, both in cortex and hippocampus. Scopolamine/DFP treated animals showed no significant differences from animals treated with DFP and vehicle alone, compatible with the short half-life for this muscarinic blocker [Kobayashi S. et al. (1981) Arzneimittelforschung 31(4):679-90]. FIG. 3 summarizes these values in a graph, demonstrating that M1 muscarinic blockade selectively accelerates the decrease in cortical ACHE. Alternatively, the M1 muscarinic blockade prevents recovery through ACHE synthesis following DFP inhibition.

1B. Sucrose Gradient

To gain more information on the composition of ACHE isoforms following these cholinergic manipulations, we performed sucrose gradient centrifugations and assessed the sedimentation values of cortical AChE.

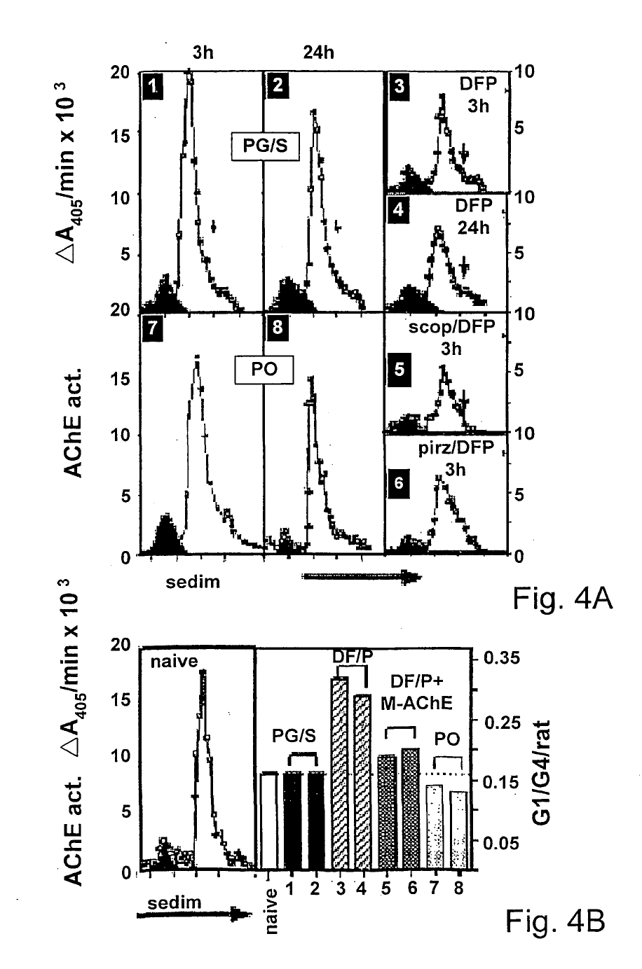

As expected, the majority of AChE activity in cortical homogenates sedimented as globular tetramers (G4) with sedimentation coefficients of 9-10 S (FIG. 4). This suggested that these enzyme molecules consist of multimeric AChE-S monomers [Sternfeld, M. et al. (2000) id ibid.]. A smaller monomeric G1 fraction in these gradients varied in size between the different samples. This fraction may contain some as-yet monomeric AChE-; it should also include all of the ACHE-R monomers present in each sample, since these monomers cannot aggregate, having no cysteine residues at their C-terminus [Soreq and Seidman (2001) id ibid.]. Therefore, the previously observed feedback response to stress or anti-AChE exposure should, in principle, increase the G1/G4 ratio. There was no significant difference in this ratio under vehicle injection (FIG. 4B), suggesting maintenance of AChE-R: AChE-S homeostasis following mild stressors in the rat cortex. In contrast, G1:G4 ratios increased by approximately two-fold at both 3 and 24 h following DFP injection, an increase which was prevented by either scopolamine or pirenzypine co-injection (FIG. 4). Finally PO, which blocks AChE, and both the excitatory M1 and the inhibitory pre-synaptic M2 receptors, reduced G1/G4 levels. These findings are compatible with the hypothesis that ACHE inhibition under impaired muscarinic neurotransmission cannot induce the characteristic AChE-R accumulation. This further implied that the alternative splicing shift that precedes AChE-R overproduction depends on muscarinic responses.

1C. Detection of AChE Expression by In Situ Hybridization

To evaluate AChE expression at the mRNA level, fluorescent in situ hybridization (FISH) analysis was performed. FIG. 5 presents examples for hippocampal FISH results, which show massive increases in both AChE-S and AChE-R mRNA labeling 3 hours following PG/S vehicle treatment as well as dramatic impairments in this response under ACHE and muscarinic blockade (DFP and scopolamine treatment, respectively).

Because each hybridization probe is likely to have distinct properties, FISH data were compared for each probe alone. AChE-S labeling is displayed in FIG. 5A, while AChE-R labeling is displayed in FIG. 5B.

Quantification of the fluorescent rhodamine signal in confocal images of cortical and hippocampal neurons demonstrated that high AChE-S mRNA levels were maintained in stressed animals, while in DFP-treated the mRNA levels were significantly reduced. For example in the hippocampal CA3 region, 81.2±9.9 compared to 29.8±5.5 arbitrary fluorescence units (AFU), respectively. The prominent DFP-induced AChE-S expression (54.2±7.6 AFU) was suppressed by both muscarinic blockers (35.1±10 AFU in scopolamine-treated, 30.4±4.9 AFU in pirenzepine-treated rats). In contrast, AChE-S elevation by vehicle injection-induced stress (44.8±7.4 AFU) was not affected significantly (39.6±3.3 AFU scopolamine, 39.1±5.2 AFU pirenzepine). This comparison showed conspicuous increases in AChE-S mRNA under PG/S with or without scopolamine, a somewhat lower increase under PG/S plus pirenzepine and reduction to basal levels 24 hours after PG/S injection. DFP prevented this AChE-S increase, while DFP plus scopolamine or pirenzepine did not, suggesting that muscarinic antagonists interfere with the feedback response to organophosphate anti-AChEs in the rat brain.

AChE-R mRNA analysis (FIG. 5B) yielded a different picture, with a relatively less dramatic increase in response to PG/S injection, yet more limited increase under both PG/S plus scopolamine or PG/S with pirenzepine but persistence of considerable increase by 24 hours. In DFP-treated rats, AChE-R mRNA accumulation was as prominent as under PG/S; but both DFP plus scopolamine and DFP plus pirenzepine blocked this increase. As ACHE-R mRNA accumulation reflects novel transcripts that underwent an alternative splicing shift, this analysis suggested that DFP enhanced such a shift in a manner suppressible by muscarinic antagonists. This would explain the mirror images of the FISH results with the two variant probes (FIG. 5) and is compatible with the DFP-induced increase in G1 AChE monomers, which was likewise suppressible by muscarinic blockers (FIG. 4).

Quantitative evaluation of the FISH data for the parietal cortex and hippocampal CA1 and CA3 neurons is presented in FIG. 6. The brain region comparison showed parallel although quantitatively distinct outcomes for the three different brain regions. Control AChE-S mRNA levels demonstrated faster retrieval, as compared with persistent >24 hr increases in AChE-R mRNA labeling following the mildly stress-inducing PG/S injection.

ACHE-S mRNA is the primary brain transcript in all mammals studied so far, rats included, whereas the level of AChE-R transcript is relatively low under normal conditions. The three-fold increase in cortical and hippocampal AChE-S mRNA levels represented a rather dramatic response to the mild PG/S stress stimulus. Its reduction after 24 hours reflected the transient nature of this phenomenon. In contrast, the PG/S induced increase in AChE-R mRNA was more limited (2, 2.5 and 1.7-fold in cortex, CA1 and CA3, respectively) yet long-lasting, being largely sustained by 24 hr (FIG. 6). The limited inter-animal variability and the mirror image changes in AChE-S compared to AChE-R mRNA labeling under pirenzepine with DFP reinforced the validity of these findings.

While AChE-S and AChE-R mRNA labeling values could be compared on their own, comparing the changes in the R:S ratios provided valuable information, presented in FIG. 7. This analysis demonstrated a conspicuously higher R:S ratio under DFP treatment as compared to PG/S alone, which was reminiscent of compatible with the gradient data. It also showed that PO, the combined AChE inhibitor-M1/M2 muscarinic blocker, induced a totally different effect from that of DFP in that it lowered the R:S ratio in comparison to DFP or PG/S administration, demonstrating massive AChE-S mRNA overproduction but no shift in alternative splicing under PO treatment. This further emphasized the long-lasting increase in the R:S ratio, under anticholinesterases, especially in cortex and CA1 neurons.

### Example 2

Alternative Splicing and Neuritic Translocation Under Short Term Treatment and Long Term Neuronal Hypersensitivity

AChE modulations provide an appropriate case study for exploring long-term stress effects. Chemical, psychological and physical stresses all shift the 3′ alternative splicing from the primary mRNA product, encoding the AChE-S multimeric protein, to the normally rare, AChE-R mRNA transcript, which yields soluble AChE-R monomers [Soreq and Seidman (2001) id ibid]. To address the cellular dynamics of these processes and their physiological consequences, the inventors examined neuronal distributions of the two AChE mRNA splice variants, which have distinct 3′ regions (FIG. 8A). Synaptic ACHE-S mRNA, terminates at the 3′ end with exon 6, whereas the stress-induced AChE-R mRNA variant terminates with pseudo-intron 4 and exon 5. A comprehensive search in the NCBI GenBank and EST databases revealed several AChE-S mRNAs but only a single AChE-R mRNA of rodent brain origin (GenBank Accession No. X70141), attesting to the scarcity and/or instability of neuronal AChE-R mRNA under normal conditions. To study changes in ACHE gene expression at a subcellular level, double4abeling fluorescent in situ hybridization (FISH) of specific AChE mRNA splice variants was followed by confocal microscopy-based image analysis. Labeling efficiencies likely depend on probe sequences, but subcellular distributions could be reliably compared for single transcripts in different cells and conditions.

2A. AChE-S mRNA Resides in Extended Neurites

Cultured PC12 cells [N. Galyam et al. (2001) Antisense Nucleic Acid Drug Dev 11, 51-7], primary cultures of mouse cerebellar neurons [M. Schramm et al. (1990) Proc Natl Acad Sci USA 87, 1193-7], and paraffin-embedded pyramidal neurons of the prefrontal cortex [Kaufer et al. (1998) id ibid.] all displayed larger fraction of AChE-S mRNA transcripts in neuronal processes than that of AChE-R mRNA (FIGS. 8B-D). For example, in PC12 cells, red Cy3 emission of labeled AChE-S mRNA and green emission of fluorescein-labeled AChE-R mRNA yielded yellow cell bodies. Nevertheless, both the peripheral cytoplasm and extended neurites displayed predominantly more of the AChE-S mRNA transcript (red) than ACHE-R mRNA (green, FIG. 8B). To test whether labeling properties were the cause, we reversed the labeling mode of the two probes. In primary cultures of cerebellar neurons (postnatal day 5), neurites presented predominant green Cy2-streptavidin conjugates bound to AChE-S mRNA, whereas cell bodies also emitted rhodamine red ACHE-R mRNA fluorescence of anti-DIG antibodies, yielding a yellow color (FIG. 8C). Also, both cell types displayed nuclear labeling of AChE-R but not AChE-S mRNA (FIG. 8B, C and data not shown). In paraffin-embedded brain sections from naive mice, cortical pyramidal neurons presented dispersed AChE-S mRNA labeling (green) throughout the processes, while AChE-R mRNA (red) was localized to the cell body. In addition, the perikaryal cytoplasm exhibited punctuated concentrations of AChE-R mRNA alternating with double-labeled (yellow) regions, whereas neurites included AChE-S mRNA (green) with focal spots harboring both transcripts (yellow, FIG. 8D). The neuritic contents in cultured PC12 cells and cerebellar neurons and in prefrontal cortex neurons in vivo were 22±3, 28±4 and 28±7% for ACHE-S mRNA but only 10±2, 7±2 and 11±6% for AChE-R mRNA, respectively (FIG. 8). AChE-S mRNA thus appeared to adopt preferential neuritic localization inherent to this transcript.

2B. Corticosterone Elevates AChE-R mRNA in Cultured PC12 Cells

The human ACHE gene includes a glucocorticoid responsive element (GRE) at about 17 kb upstream from the transcription initiation site. In humans, a GRE-adjacent deletion induces constitutive overexpression and anti-AChE hypersensitivity [Shapira et al. (2000) id ibid.], which suggested a physiologically significant role for glucocorticoids in regulating both neuronal ACHE gene expression and hypersensitivity. The inventors therefore compared PC12 cells treated with 10 mM corticosterone (10 mM in ethanol diluted 1:1000, 6 hrs) to cells treated with vehicle alone (0.1% ethanol, 6 hrs). Corticosterone induced marked increases (30-50% higher labeling intensities within 24 hrs, p<0.05, 2-tailed Student's t-test) in both splice variants as compared to the vehicle-treated cells. Twenty four hours following corticosterone treatment, catalytic activity measurements confirmed an increase of 25±14% (p<0.05) in the acetylthiocholine hydrolytic capacity of PCl₂ cells AChE. This supported the notion that the distal enhancer GRE mediates at least part of the neuronal AChE stress response. FIGS. 9A-D displays examples of the corticosterone-induced elevation of AChE-S and ACHE-R mRNAs in PC12 cells. AChE-S mRNA-labeled area remained essentially unchanged under control conditions (distance from nuclear border to cell margins=37±13 mm) or corticosterone (34±13 mm). In contrast, AChE-R labeling extended a smaller distance from the nucleus, 25±9 mm under control conditions, which increased to 33+17 mm distance under corticosterone treatment (p<0.05). This indicated that AChE-S mRNA was evenly distributed throughout the cell body, whereas AChE-R mRNA, which initially assumed a perinuclear distribution, migrated into cellular periphery when overproduced. Whether these glucocorticoid effects are direct, or are mediated by other elements, is yet to be clarified.

2C. Physical, Chemical and Psychological Stresses, Facilitate Long-Lasting Neurite Translocation of AChE-R mRNA In Vivo

Because of the secretory nature of ACHE-R, we used FISH detection of the intracellular AChE-R mRNA transcript to assess the expression levels of this variant in vivo. Dorsal hippocampal neurons of naive FVB/N mice express extremely low levels of AChE-R mRNA under normal conditions (FIG. 10A). Two days following the stress of surgical implantation of a microdialysis probe [Erb, C. et al. (2001) id ibid.], neuronal AChE-R mRNA levels increased considerably, especially in neurites, with the distance of neurite labeling increasing from 2.0±0.3 to 5.1±1.0 mm per cell (FIG. 10C, p<0.0005). Injection through the cannula with the AChE inhibitor neostigmine (125 nmol) resulted within 25 minutes in a more extensive translocation of AChE-R mRNA (to 8.5±1.2 mm I cell, FIG. 10E). Untreated chemically hypersensitive AChE-S transgenic mice displayed significantly longer AChE-R mRNA labeled neurites than their strain-matched FVB/N controls (9±1 mm from the nucleus, FIGS. 10B, H, p<0.0005), consistent with previous findings [Shapira, M. et al. (2000) Hum. Mol. Genet. 9, 1273-1281]. Cannula implantation failed to increase labeling further (10.5±1 mm, FIG. 10D), but ACHE-R mRNA reached dendrite distances of 15+2 mm under neostigmine (FIG. 10F), significantly longer than either similarly treated non-transgenic animals, or cannulated transgenics (in both cases p<0.0005).

The reported rates of mRNA dendritic transport range from 10-20 mm/hr [Davis L., et al. (1990) J Neurosci 10, 3056-68] to 300-360 mm/hr [Wallace, C. S. et al. (1998) J Neurosci 18, 26-35]. In the present study, assuming a constant rate, AChE-R mRNA traveled a distance of 8±5 mm/hr in anticholinesterase-treated FVB/N mice, which increased to 14±7 mm/hr in similarly treated hAChE-S transgenic mice. This rate is consistent with the lower range estimate, suggesting that slow loading of the dendritic tree may provide accessible storage of AChE-R mRNA for local translation and secretion [O. Steward and E. M. Schuman (2001) Annu Rev Neurosci 24, 299-325]. Combined with its apparent durability as reflected in ACHE-R mRNA sheltering from antisense destruction, this analysis predicted long-lasting post-stress neuritic presence of this transcript in vivo. To test this prediction, we subjected FVB/N mice to 4 consecutive days of forced swim (two 4 min swim sessions per day). In naive mice, cerebellar granule neuron processes were loaded with 28±12% of the cellular AChE-S mRNA but only 9±2% of the AChE-R mRNA content, reaching up to 15 mm from the nuclear border (FIG. 11A). Two weeks post-stress, a considerably larger fraction (24±7%) of ACHE-R mRNA translocated into neurites of cerebellar granule neurons, which displayed patches of concentrated AChE-R mRNA in both the cell bodies and processes (FIG. 11B).

2D. Long-Term AChE-R mRNA Overproduction is Associated with Hippocampal Hypersensitivity

In non-transgenic mice, hippocampal AChE-R mRNA signals are generally limited to the granular layer [Kaufer et al. (1998) id ibid.]. AChE-R mRNA levels remained considerably higher than baseline 4 weeks after 4 consecutive days of forced swim or 3 day exposure to very low diisopropylfluorophosphonate (DFP) levels (0.1 mg⁻¹kg⁻¹day⁻¹, ip., 0.08 LD₅₀). Pre-stressed or pre-exposed animals presented high intensity labeling in the hippocampal CA1 region, dentate gyrus and dendritic layer (FIGS. 12A-F and data not shown), predicting modified composition of neuritic AChE variants both after stress or low-level exposure to antichohnesterases.

Released neuronal ACh binds to both pre- and post-synaptic receptors and is assumed to serve as a modulatory neurotransmitter and set the response level of the neuronal network to incoming stimuli [Gray, R. et al. (1996) Nature 383, 713-6]. This involves electrophysiological mechanisms that are only partly understood, but presumably include on-site hydrolysis of acetylcholine by AChE-S. To test whether the substitution of neuritic AChE-S with AChE-R compromises the capacity to confront stressful cholinergic stimuli, the inventors measured the excitatory synaptic transmission in the CA1 area of the hippocampus. Population field potentials (pfps) were recorded in hippocampal slices [Friedman, A. et al. (1998) J Physiol Paris 92, 329-35] from naive and pre-stressed animals one month after exposure to stress. In sections from naive animals, the anticholinesterase physostigmine induced 20-80% increase (42±15%, n=4) in the extracellularly recorded amplitude of the evoked population spikes response to stratum oriens stimulation. This response was 70% reversible following the addition of the muscarinic antagonist atropine to the bathing solution (FIG. 12G). In non-stimulated sections from animals tested one month following 4 days of repeated stress, the mean pfp amplitude was similar to that of non-stimulated controls. However, exposure to physostigmine of sections from previously stressed mice resulted in a 12-fold larger increase over baseline in population spike amplitudes than that observed in non-stressed animals. Atropine administration reversibly blocked this evoked response by over 90%, much more effectively than its capacity to block field potentials in the control brain (FIG. 12H). As in control mice [Friedman et al. (1998) id ibid.], the evoked pfp was blocked by the NMDA and AMPA antagonists aminophosphonopentanoic acid (APV) and dinitroquinoxalinedione (DNQX), respectively, attesting to its glutamatergic nature. Exposure to low DFP doses similarly induced long-term exaggerated hypersensitivity of synaptic responses to either stimulatory or inhibitory cholinergic agents (data not shown). While the detailed mechanisms leading to this hypersensitivity await further investigation, the general phenomenon is consistent both with the chemical hypersensitivity phenotype of the AChE-overproducing transgenic mice [Shapira (2000) id ibid.] and with the changes in ACHE gene expression in FVB/N mice. This tentatively attributes a role for stimulus-induced AChE-R overexpression and neuritic translocation in the long-known neuronal hypersensitivity that follows stressful experiences [Antelman, S. M. et al. (1980) Science 207, 329-31] for which there has been no mechanistic explanation.

### Example 3

AChE-R Forms Neuronal Complexes with PKCβII and Its WD Carrier RACK1

The present inventors have previously showed by yeast two hybrid system, that the ARP peptide derived from the readthrough acetylcholinesterase variant (AChE-R) interacts with the cognate receptor for activated kinase C (RACK1) [PCT application IL00/0031].

In order to test whether ARP further promotes triple complex formation with PKCβII and RACK1 in mammalian cells, a vector designated PGARP, that encodes a fusion protein between green fluorescent protein (GFP) and ARP1 under the CMV promoter was used. When transfected into COS cells, which do not express AChE, anti-ARP antibodies immunodetected GARP expression in cell homogenates. Anti-GFP antibodies were ineffective in non-transfected cells but immunoprecipitated GARP, RACK1 and PKCβII from homogenates of GARP transfected COS cells (as shown by FIG. 13A).

3A. AChE-R Promotes Triple Complexes with RACK1 and PKCβII in Native PC12 Cells

Both COS and PC12 cells express RACK1 and PKCβII constitutively, whereas only PC12 expresses AChE-R, as observed in immunoblots of the soluble fraction of cell homogenates (FIG. 13B1). Antibodies targeted to the N-terminal domain of AChE co-immunoprecipitated both PKCβII and RACK1 in PC12 but not COS cells, supporting the notion of tight binding for AChE-R/RACK1/PKCβII in these PC12 cell complexes (FIG. 13B2).

3B. Stress Induces Neuronal Accumulation of Immunoreactive AChE-R and RACK1

The in vivo relevance of AChE-R/RACK1 interactions was explored in normal and post-stress mouse brain. Immunoreactive RACK1 was observed in the cytoplasm and closely proximal processes of pyramidal neurons, in layers 3 and 5 of the frontal and parietal cortex, in both superficial and deep layers of the piriform cortex, and in regions CA1 and CA3 of the hippocampus. A subset of these neurons also overexpresses AChE-R under acute psychological stress (FIG. 14 and data not shown). Stress-induced increase of RACK1 was seen in parietal cortex layer 5 (compare FIGS. 14C to 14E and 14G to 14I). Unlike RACK1, ACHE-R antibodies also stained cells with glial morphology. Also, in some regions, such as hippocampal CA1, RACK1 staining formed an almost continuous pattern, whereas AChE-R was localized to a subset of pyramidal neurons. For both AChE-R and RACK1, uneven perikaryal accumulation and increased neurite labeling were observed under stress (FIG. 14).

3C. Transgenic ACHE-R Overexpression Elevates Brain RACK1 Levels and Intensifies the Formation of Neuronal PKCβII Clusters

Hippocampal homogenates from ACHE-R overexpressing transgenics [Sternfeld et al. Proc Natl Acad Sci USA, 97, 8647-52 (2000)] were tested to investigate whether ACHE-R overproduction would modulate the levels, properties and/or neuronal localization of its partner proteins RACK1 and PKCβII. The results show that the hippocampal homogenates displayed significant increases (compared to the levels in control FVB/N stress-prone mice) of neuronal AChE-R and RACK1, as well as a faster migrating PKCβII band that was only faintly detected in the hippocampus of a control animal (FIG. 15A). xxxx

Of the three target proteins, RACK1 and AChE-R appeared more widely distributed and could be detected in numerous brain regions (FIG. 15B and data not shown). In the brain of AChE-R transgenics, AChE-R overexpression was particularly conspicuous in neuron groups showing punctuated PKCβII staining (FIGS. 15B-D). PKCβII labeling, in contrast, appeared higher than control levels in only a fraction of the AChE-R overexpressing subregions. Finally, RACK1 staining was intensified in the ACHE-R expressing hippocampal CA1 and dentate gyrus neurons, and less prominent in the parietal cortex. This result suggested that the AChE-R/RACK1/PKCβII interactions facilitated the intracellular retention of the secretory AChE-R protein.

3D. Diverse Subcellular Distributions of PKCβII

In control mice, PKCβII antibodies displayed diffuse staining [Weeber et al. (2000) J Neurosci, 20, 5906-14.] in sub-regions of layers 5, 6 in the cortex, in the stratum oriens and stratum radiatum layers of the hippocampus CA1 field, in the striatum matrix, and in the substantia nigra pars reticulata. Axonal bundles including the nigro-striatal tract were also labeled (data not shown). Another and novel staining pattern consisted of dense PKCβII clusters in neuronal perikarya and in the axonal stems. This punctiform pattern appeared in upper layers of the parietal, temporal and piriform cortex, dorsal striatum, basolateral amygdala, hippocampal CA1 and lateral septum. In general, cells in AChE-R transgenic mice that displayed prominent AChE-R labeling were positive for RACK1 and presented PKCβII punctiform staining (FIGS. 15B to 15E). AChE-R labeling in cells where in control mice included AChE-R, RACK1 and punctated PKCβII, was not intensified in AChE-R transgenic mice. These included neurons in the globus pallidus, substantia nigra, superior culliculus, medial septum and diagonal band (FIGS. 15B to 15E and data not shown). Other neurons were positive for AChE-R staining in the control mice, and yet more so in AChE-R transgenic mice, but had no PKCβII punctiform staining. These resided in the lateral and ventro-medial hypothalamus, central nucleus of the amygdala, the hippocampal dentate gyrus, ventro-lateral thalamus, and the Edinger-Westphal nucleus (FIGS. 15C to 15D). C57B6J mice were tested for the punctated staining pattern, and a weaker but discernible punctiform signal was observed in the same cell populations as in the stress-prone FVB/N strain, used as control (data not shown).

3E. Inter-Related AChE-R/RACK1/PKCβII Distributions

In samples obtained from AChE-R transgenic mice, anti-PKCβII antibodies detected diffuse and axonal staining patterns similar to those observed in the parental FVB/N strain. However, the punctated pattern was altered. Stronger and denser clusters of PKCβII staining were located on the perikaryal circumference of a larger fraction of hippocampal CA1 neurons (FIG. 15E2). Transgenic mice overexpressing the major synaptic isoform of AChE [Beeri, R. et al. (1995) Curr Biol, 5, 1063-71] did not show such changes in PKCβII expression (data not shown), suggesting that this i7, vivo effect depended on chronic AChE-R excess and/or that it was prevented by AChE-S excess. ACHE-R staining in the transgenic brain was prominent in the cell bodies and proximal processes of many, but not all CA1 hippocampal neurons, suggesting that a specific subset of these neurons was especially amenable for such accumulation (FIGS. 15E3, 4). Sparse cells with morphology reminiscent of microglia were also positive for ACHE-R staining, both in control and transgenic animals (FIGS. 15E5, 6). Intensified labeling of perikarya and closely proximal neurites of CA1 pyramidal neurons was also observed by staining with RACK1 antibodies (FIGS. 15E3, 4).

3F. Subcellular Distributions of AChE-R, RACK1 and PKCβII was Overlapping

Confocal micrographs of upper layer neurons from the parieto-temporal cortex double labeled with antibodies against ACHE-R and RACK1 or PKCβII displayed distinct yet overlapping distributions for the three partner proteins within neuronal perikarya in compound field projections. As expected, ACHE-R labeling was conspicuously more intense in AChE-R transgenics than in FVB/N controls (compare in FIG. 16, A1, 2 to 3 and 4). The overexpression and the associated overlapping increases in the two partner proteins were reflected in different colors. FIG. 16A presents fields of double stained neurons and FIGS. 16B and 16C, enlarged single cells.

Proteins destined to be secreted are initially concentrated near the nucleus, where their processing takes place, whereas proteins that are associated with the perikaryal cytoskeleton, plasma membrane and/or proximal process structures are distributed more peripherally in the cell. In cortical neurons from control mice, both ACHE-R and RACK1 immunostaining formed peri-nuclear accumulations (compare in FIG. 16B1 to 3). In contrast, the intensity of RACK1 staining in ACHE-R transgenic mice was especially high around the perikaryal circumference (compare in FIGS. 16B5 to 2), suggesting subcellular translocation under ACHE-R excess. PKCβII staining clusters were also removed from the peri-nuclear domain in the control mice (FIG. 16C8), and showed uneven distribution in larger cellular spaces in transgenic mice (FIG. 16C11). In control mice, PKCβII patterns differed from both those of AChE-R and RACK1 in that they demonstrated both diffuse staining and punctated clusters of protein complexes, compatible with the parallel light microscopy patterns. Constitutive AChE-R overexpression further enlarged the perikaryal space occupied by AChE-R, RACK1 and PKCβII and seemed to increase the intracellular density of their complexes.

### Example 4

4A. Overexpression of Runx1/AML1 and AChE in Primary Astrocytic Tumors

Several transcription factor binding sites along the AChE promoter may potentially control its tumorigenic gene expression. The distal upstream enhancer domain harbours, among others, consensus binding sites for HNF3α/β and, c-fos (54). The proximal promoter [Shapira (2000) id ibid] and first intron [Getman, D. K. et al. (1995) J Biol Chein 270, 23511-9] both include consensus binding sequences for c-fos and Runx1/AML1 (FIG. 17A). c-fos and HNF3β were both shown to control neuronal AChE expression under psychological and chemical stresses, respectively [Kaufer et al. (1998) id ibid.; Shapira et al.(2000) id ibid.], however, their astrocytic expression was never sought. Runx1/AML1, a major hematopoietic transcription factor, is well known for its association with human leukemia [Westendorf, J. J., and. S. W. Hiebert (1999) J Cell Biochem Suppl:51-8; Perry, C., A. Eldor, and H. Soreq (2001) Leuk. Research], but has not yet been described in brain tumors. By designing in situ hybridization probes, the present inventors tested the functional relevance of these factors for controlling AChE gene expression in astrocytomas.

High resolution in situ hybridization revealed very low expression levels for the AChE-S and AChE-R transcripts in paraffin sections from grade I primary astrocytomas, consistent with the low level of AChE gene expression in non-tumor astrocytes [Karpel (1996) id ibid.]. In contrast, AChE-S and ACHE-R expression increased significantly in tumors with pathologically defined increasing aggressiveness (FIG. 17B). For the AChE-S transcript, in 5 sections from 4-5 different tumors from each grade (I-IV), the increase was especially noticeable between astrocytoma grade I and II (p<1*10⁻⁹, Student's t test), with a mild increase from grade II to III (p<0.08). For the AChE-R transcript, the increase in expression was more significant in the more advanced grades (p<0.0005 for both I to II and II to III, but 7*10⁻⁷ for III to IV). Moreover, both cfos and HNF3□ mRNAs, known to be co-expressed with AChE in neurons, did not show significant expression levels in astrocytomas, although c-fos was weakly detected in grade IV astrocytic tumors FIG. 17B). Surprisingly, Runx1/AM1 mRNA levels, detected by in situ hybridization, increased with astrocytoma aggressiveness in a similar manner to that of the ACHE mRNA transcripts. Two independent Runx1/AML1 probes revealed similar patterns in astrocytomas grade I to III (FIG. 17B and data not shown). The pattern of steep increase (p<0.0002) between grades I and II, plateau between II to III followed by a decrease (p<0.008) between m to IV, suggested a causal correlation between Runx1/AML1 and AChE gene expression. It is interesting to note that in sections from brain metastasis of malignant melanoma, ACHE-S, ACHE-R and Runx1/AML1 transcripts were overexpressed as intensively as in grade III-IV astrocytomas.

4B. Runx1/AML1 and AChE are Overexpressed in Tumor-Associated Endothelium

The gradual elevation in astrocytoma ACHE gene expression could be limited to the tumor cells or caused by soluble growth factors. In the latter case, it should also take place in non-tumor surrounding cells. To distinguish between these possibilities, we examined vascular endothelial cells from astrocytomas with increasing aggressiveness. Diffuse cytoplasmatic signals of ACHE and Runx1/AML1 transcripts increased gradually within vascular structures from astrocytoma and metastatic brain melanoma (FIG. 17C). In contrast, HNF3□ and c-fos showed negligible expression levels in vascular endothelium (data not shown), suggesting that similar elements induced Runx1/AML1 and AChE gene expression in tumor astrocytes and adjacent endothelial cells.

Image analysis was employed to characterize the AChE mRNA variants in astrocytic and endothelial cells. FIG. 18A presents, for four tumor specimens from each pathological grade, tumor grade-related increases in AChE-S, AChE-R and Runx1/AML1 transcripts, while not in c-fos or HNF3□ transcripts. AChE expression in endothelial cells depended on tumor grade (FIG. 18B), however with more limited increases in expression levels than those observed in tumor cells. AChE-S transcript increased significantly from grade I to II (p<0.0002) but not from II to III or III to IV; ACHE-R transcript increased less significantly, even from grade I to II (p<0.02), whereas Runx1/AML1 increased steeply from grade II to III (p<0.002) but presented lower differences between grades III to IV (p<0.03). Inter-specimen variations were relatively small, both in tumor cells and in vascular endothelium.

4C. Increasing AChE mRNA Splicing Shift with Tumor Aggressiveness

Both the primary ACHE-S transcript and the stress-induced AChE-R transcript increased in labelling intensity with increasing tumor aggressiveness. However, the best-fit correlation (R²=0.985) for labelling intensity values of AChE-R and ACHE-S mRNAs in each pathological grade was exponential. In contrast, endothelial cells presented a linear best-fit correlation (R²=0.987) (FIG. 18C), demonstrating steady ratios between the two transcripts regardless of tumor grade. Thus, AChE-R/AChE-S relationships were clearly distinguishable in tumor and the surrounding benign cells.

4D. Retention of Runx1/AML1 and AChES but Not AChE-R in Astrocytic Tumor Cells

Pronounced accumulation of the Runx1/AML1 protein was observed in tumor cells nuclei, with some cytoplasmatic labelling in astrocytoma tumors of particularly aggressive phenotypes and in melanoma metastases (FIG. 19A). Immunocytochemical analysis demonstrated that AChE-S exhibited increasing intensities in astrocytoma tumors, especially grade III, and was also detected in metastatic brain melanoma. In contrast, the soluble AChE-R protein was barely detectable in astrocytoma sections (FIG. 19B), while being intensively stained in metastatic brain melanoma (FIG. 19C). In summary, these results suggest that tumor-specific mechanisms thus enable ACHE-R secretion from astrocytoma but its retention in melanoma cells, while cellular AChE-S is retained in both.

4E. Runx1/AML1 together with the co-Activator p300 Facilitate AChE Production

AChE constructs including the proximal promoter and the first intron (AC6), and those including the distal enhancer domain (wtAC6), or with the HNF3□ binding site deletion (□AC6) [Shapira (2000) id ibid.] were co-transfected into COS1 cells with the full (Runx1/AML1) or truncated (tRunx1/AML1) Runx1/AML1 constructs, the latter devoid of the transcription activating domain, and with a p300 vector [Eckner, R. et al. (1994) Genes Dev. 8,869-84]. Measurement of the catalytic activity of nascent AChE was used as a reporter. AChE catalytic activity in COS-1 cells transfected with a GFP expression vector served as control. Electromobility shift assays (EMSA) showed a barely detectable association between Runx1/AML1 and consensus Runx1/AML1 binding sequences from the AChE proximal promoter (data not shown). Consistent with this, we observed only a minor increase (30±8% above baseline) in AChE activity in COS1 cells co-transfected with AC6 and Runx1/AML1, as compared with cells transfected with AC6 alone (FIG. 20). On its own, the Runx1/AML1 transcriptional co-activator p300 had no significant effect on AChE expression. However, when COS-1 cells were co-transfected with AChE, Runx1/AML1 and p300, they exhibited up to 3-fold enhanced AChE expression. This effect was further exacerbated (up to 6-fold increase) in the presence of the ACHE distal enhancer domain (Student's t test p<0.035). The p300-Runx1/AML1 (or p300-tRunx1/AML1) enhancement of AChE expression, as compared to Runx1/AML1 alone was also pronounced in the presence of the AChE mutated distal domain (□AC6) (p=0.025 and p=0.03, respectively). These findings suggested functionally relevant effects, in controlling AChE gene expression for p300 in conjunction with the Runx1/AML1 DNA binding domain in the proximal promoter. Because the CMV promoter may also be controlled by p300, we tested if the p300-induced difference in AChE gene expression did not merely reflect an increase in Runx1/AML1 levels under these co-transfection conditions. However, immunoblot experiments revealed no apparent change in Runx1/AML1 levels under co-transfection with p300 (data not shown). Therefore, we conclude that p300 and Runx1/AML1 act synergistically on the AChE promoter, enhancing AChE gene expression.

4F. The AChES C-Terminal Peptide (ASP) Facilitates Runx1/AML1 Nuclear Localisation

The apparently distinct expression pattern of the two AChE variants in astrocytic tumors called for testing the biological properties of their distinct C-terminal peptides. To this end, we constructed three GFP fusion expression vectors with downstream AChE C-terminal peptides. These included the AChE “Synaptic” peptide (GASP), with the conserved CSDL motif shown previously to be essential for subunit assembly [Velan, B. et al. (1994) J Biol Chem 269:22719-25], a mutated GASP (mGASP) in which a frame-shift mutation changed the final eight amino acids of ASP, abolishing this motif, and the ACHE-R “readthrough” peptide (GARP) (FIG. 21A). COS1 cells transfected with a non-fused GFP expression vector showed widespread whole cell labelling. However, both normal and mutated GASP primarily located to the nucleus, whereas GARP formed a peri-nuclear cytoplasmic “cap” in what seemed to be the Golgi apparatus (FIG. 21B), suggesting that the ASP element was the cause for nuclear localization. To test if the nuclear localization of GASP reflected a global change in nuclear transport mechanisms, we performed co-transfection experiments with Runx1/AML1. When transfected alone, the Runx1/AML1 protein showed nuclear localisation in 70% of transfected COS1 cells, while in the remaining 30% it presented cytoplasmic localisation. However, when co-transfected with the, GASP expression vector, Runx1/AML1 resumed exclusive nuclear localisation (FIG. 22), suggesting that ASP, and perhaps AChE-S as well, may facilitate the nuclear localisation of Runx1/AML1. Further studies will be required to find out if this effect is indirect or if it involves direct ASP-Runx1/AML1 interactions.

1. A method for evaluating an effect on the nervous system of a test drug, wherein said method comprises the steps of:
(a) providing the following groups of animals: (i) at least one control group; and (ii) a test group;
(b) at time zero, injecting each of the animals of the control group with a carrier and each of the animals of the test group with said test drug contained within said carrier;
(c) after a predetermined period of time from time zero, injecting the animals of the control group with an agent selected from:
said carrier, an irreversible acetylcholinesterase (AChE) blocker or an ACHE, M1 and M2 blocker; and
injecting the animals of the test group with the same agent used for the control group;
(d) after a first predetermined period of tune from step (c), sacrificing a number of animals from each of said control and test groups and dissecting their brains;
(e) after a second predetermined period of time from step (c), sacrificing the remaining of animals of each group and dissecting their brains;
(f) subjecting the dissected brains to at least one analytical procedure for assessing AChE catalytic activity or isoform variance, or any combination thereof;
(g) generating a drug profile from the results of the analytical procedures performed in (f) for each of the control and test groups; and
(h) comparing the profile of the test group with the profile displayed by the control groups;
whereby
increased expression in the brain of AChE mRNA transcripts, with or without a shift in the alternative splicing of said transcripts from the primary S to the normally rare R variant indicates a distinct feedback response to said test drug;
an increase with time in the catalytic activity of brain AChE, with or without an increase in the globular G1 ACHE monomers to G4 tetramers reflects the outcome of such feedback at protein level; and
any diversion from the brain response of the control to said irreversible ACHE blocker reflects an impairment in muscarinic neurotransmission resulting from the exposure to said test drug.

2. A method according to claim 1, wherein in step (b), the following three control groups are employed:
group (ii-a), wherein at time zero each of the animals is injected with a carrier, providing baseline control;
group (ii-b), wherein at time zero each of the. animals is injected with a general non-selective muscarinic receptor blocker; and
group (ii-c), wherein at time zero each of the animals is injected with a M1 blocker.

3. A method according to claim 1 or 2, wherein said predetermined period of time from time zero is from 45 to 90, preferably 45 minutes, said first predetermined period of time from step (c) is from 2 to 4, preferably 3 hours, and said second predetermined period of time from step (c) is from 24 to 36, preferably 24 hours.

4. A method according to any one of claims 1 to 3, wherein said test animals are rats.

5. A method according to any one of claims 1 to 4, wherein said general non-selective muscarinic receptor blocker is scopolamine.

6. A method according to any one of claims 1 to 5, wherein said selective M1 blocker is pirenzepine.

7. A method according to any one of claims 1 to 6, wherein said AChE blocker is DFP.

8. A method according to any one of claims 1 to 7, wherein said AChE, M1 and M2 blocker is octylpyridostigmine (OP).

9. A method according to any one of claims 1 to 8, wherein said carrier is propylene glycol/saline.

10. The method according to any one of the preceding claims, wherein the analytical procedures are selected from the group consisting of determining AChE activity, determining AChE S and/or R isoforms ratio and determining AChE-S and/or -R mRNA variants tissue expression and distribution, by any suitable means.

11. The method according to claim 10, wherein the AChE isoform ratio is determined by means of a sucrose gradient.

12. The method according to claim 10, wherein AChE isoform tissue expression is determined by any one of in situ hybridization utilizing isoform specific probes, and immunohistochemistry utilizing isoform specific antibodies.

13. The method according to any one of the preceding claims, wherein said test drug is selected from the group of drugs for the treatment of anxiety conditions, post-traumatic stress, Alzheimer's disease, muscle malfunctioning, neurodegenerative disorders, damage resulting from exposure to xenobiotics, panic, neuromuscular disorders, Parkinson's disease, Huntington's chorea, muscle fatigue, multiple chemical sensitivity, autism, multiple sclerosis and Shorgren's disease.

14. The method according to any one of the preceding claims, wherein the generation of said drug profile further comprises the evaluation of an astrocyte oncogenesis potential of said drug.

15. The method of claim 15, wherein said evaluation comprises the steps of:
(a) treating an animal with said test drug;
(b) dissecting the brain of said animal;
(c) contacting said dissected brain with detectable means for determining the expression of Runx1/AML;
(d) comparing the level of expression of Runx1/AML in said brain with the level of expression of the same gene in a brain tumor sample.

16. The method according to any one of the preceding claims, wherein said test drug is selected by a screening method for a candidate drug that is a modulator of the expression of any one of ACHE variants and isoforms, wherein said screening method comprises the steps of:
(a) contacting a cell, preferably a neuron, with a test drug under suitable conditions;
(b) determining the effect of said drug on an end-point indication, wherein said effect is the capacity of a drug to modulate the expression and distribution of at least one of the AChE isoforms.

17. The method of claim 16, wherein the endpoint indication is the translocation of an AChE isoform within the cell.

18. The method of claim 17, wherein said cell is a neuron and said translocation is from the cell body towards its processes.

19. The method of claim 18, wherein said AChE isoform expression and distribution is determined by in situ hybridization, whereby the detection of AChE expression and distribution is performed with AChE nRNA variant specific probes.

20. The method of any one of claims 16 to 19, wherein the cells to be treated are selected from the group consisting of PC12 cells and primary cerebellar neurons or any AChE-expressing primary neuron or established cell line.

21. The method according to claims 1 to 15, wherein said test drug is selected by a screening method for a candidate drug aimed at affecting central nervous system properties which is a modulator of the interaction between AChE-R/RACK1/PKC, which screening method comprises the steps of:
a. providing a reaction mixture comprising the AChE-R variant of AChE or any functional fragment thereof, the cognate receptor for activated kinase C (RACK1) and the protein kinase C βII (PKCβII);
b. contacting said mixture with a test drug under suitable conditions for said interaction; and
c. determining the effect of the test drug on an end-point indication, wherein said effect is indicative of modulation of said interaction by the test drug.

22. The method according to claim 21, wherein said modulator inhibits or enhances the interaction between AChE-RIRACK1/PKC.

23. The method according to claim 21, wherein said reaction mixture is a cell mixture or a cell-free mixture.

24. The method according to claim 23, wherein said reaction mixture optionally further comprises solutions, buffers and compounds which provide suitable conditions for interaction between AChE-R/RACK1/PKC and the detection of an end-point indication for said interaction.

25. The method according to claim 22, whereby modification of said end-point indicates modulation of the interaction between AChE-R/RACK1/PKC by said test drug.

26. The method of claim 23, wherein the reaction mixture is a cell-free mixture.

27. A method according to claim 26, wherein said screening method comprises the steps of:
a. providing a cell free mixture comprising the ACHE-R variant of ACHE or any functional fragment thereof, RACK1 and PKCβII;
b. contacting said mixture with a test drug under conditions suitable for an in vitro interaction; and
c. determining the effect of the test drug on co-precipitation of PKCβII and RACK1 with the AChE-R or fragment thereof as an end-point indication, whereby the absence or increase of said co-precipitation indicates modulation of formation of a complex between AChE-R/RACK1/PKC by the test drug.

28. The method according to claim 27, wherein said cell-free mixture comprises any one of ACHE-R variant of AChE or any functional fragment thereof, RACK1 and PKCβII, which are provided as a purified recombinant protein or as a cell lysate of cell expressing said proteins.

29. The method according to claim 28, wherein said AChE-R variant of AChE, is a fusion protein comprising ACHE-R or functional fragment thereof and any one of GST (Glutathion-S-Transferase) and GFP (Green Fluorescent Protein).

30. A method according to claim 22, wherein said reaction mixture is a cell mixture.

31. A method according to claim 30, wherein said cell mixture is a transfected cell culture.

32. A method according to claim 31, wherein said test drug is selected by a screening method for a candidate substance which is a modulator of the interaction between AChE-R/RACK1/PKC, which screening method comprises the steps of:
a. providing transfected cell culture expressing the AChE-R variant of AChE or functional fragment thereof, the cognate receptor for activated kinase C (RACK1) and the PKCβII;
b. contacting said transfected cell culture with a test substance;
c. detecting the interaction between AChE-R/RACK1/PKC in the presence of the test substance/drug by searching for an end-point indication, whereby inhibition of said end-point indicates inhibition of complex formation between AChE-R/RACK1/PKC by said test drug.

33. The method according to claim 32, wherein said transfected cell is transfected by:
a. an expression vector comprising a nucleotide sequence coding for the AChE-R variant of AChE or a functional fragment thereof;
b. optionally, constructs comprising a nucleic acid sequence coding for any one of the cognate receptor for activated kinase C (RACK1) and the PKCβII.

34. The method according to claim 33, wherein the end-point indication is the subcellular translocation of catalytically active PKCβII, which can be detected by a visually detectable signal.

35. The method according to claim 28, wherein the end-point indication is co-precipitation of PKCβII and RACK1 with the ACHE-R or functional fragment thereof leading to a detectable signal, whereby modification of said detectable signal in the presence of the test drug indicates modulation of the formation of a complex between AChE-R/RACK1/PKC by said test drug.

36. The method according to any one of claims 28 to 30, wherein said recombinant cell is a mammalian cell.

37. The method according to any one of claims 16 to 31, wherein said test drug is selected from the group consisting of: protein based, carbohydrates based, lipid based, nucleic acid based, natural organic based, synthetically derived organic based, antibody based and metal based substances.

38. The method according to claim 32, wherein said protein or antibody based substance is a product of a combinatorial library.

39. A system for assaying a drug for its effect on the central nervous system, said system comprising:
groups of test animals being at least one treated control group and an identical number of corresponding test groups;
a carrier, a general non-selective muscarinic receptor blocker in said carrier, a selective muscarinic receptor 1 (M1) blocker in said carrier and means for injecting the same into said animals;
an acetylcholinesterase (ACHE) blocker and means for injecting the same into said animals;
an AChE, M1 and M2 blocker, and means for injecting the same into said animals;
means for sacrificing said animals and for dissecting their brains;
means for assessing AChE catalytic activity or AChE isoform variance, or any combination thereof in said dissected brains.
